SEQUENCE LISTING (82 sequences)

SEQ ID NO: 1 - DNA, 18 bp, Artificial sequence
  Single strand DNA oligonucleotide
ctccgtggctggttttct

SEQ ID NO: 2 - DNA, 20 bp, Artificial sequence
  Single strand DNA oligonucleotide
ccgatcccagactccagaat

SEQ ID NO: 3 - DNA, 20 bp, Artificial sequence
  Single strand DNA oligonucleotide
tgacatggaatcctgacgtg

SEQ ID NO: 4 - DNA, 18 bp, Artificial sequence
  Single strand DNA oligonucleotide
gagctgaccaagggggat

SEQ ID NO: 5 - DNA, 18 bp, Artificial sequence
  Single strand DNA oligonucleotide
agtgcctgaagcccacac

SEQ ID NO: 6 - DNA, 20 bp, Artificial sequence
  Single strand DNA oligonucleotide
acccagatatgcccccttag

SEQ ID NO: 7 - DNA, 16 bp, Artificial sequence
  Single strand DNA oligonucleotide
gttgggcggtgcttgc

SEQ ID NO: 8 - DNA, 23 bp, Artificial sequence
  Single strand DNA oligonucleotide
aagaaagcgaataaagatgaggc

SEQ ID NO: 9 - DNA, 23 bp, Artificial sequence
  Single strand DNA oligonucleotide
tgcctcctgaattaactgctttt

SEQ ID NO: 10 - DNA, 23 bp, Artificial sequence
  Single strand DNA oligonucleotide
acccctgagaggtggtaccttac

SEQ ID NO: 11 - DNA, 21 bp, Artificial sequence
  Single strand DNA oligonucleotide
gcacacacctggctaaggcca

SEQ ID NO: 12 - DNA, 19 bp, Artificial sequence
  Single strand DNA oligonucleotide
caaggtgccagctccacaa

SEQ ID NO: 13 - DNA, 20 bp, Artificial sequence
  Single strand DNA oligonucleotide
cccaccccacacattttcat

SEQ ID NO: 14 - DNA, 22 bp, Artificial sequence
  Single strand DNA oligonucleotide
gaatccttcatctcacccacct

SEQ ID NO: 15 - DNA, 24 bp, Artificial sequence
  Single strand DNA oligonucleotide
ctggcaaattcagagaccaactta

SEQ ID NO: 16 - DNA, 22 bp, Artificial sequence
  Single strand DNA oligonucleotide
gtgttgggattacaggcatgag

SEQ ID NO: 17 - DNA, 21 bp, Artificial sequence
  Single strand DNA oligonucleotide
ttagcatgaggcagttttggg

SEQ ID NO: 18 - DNA, 22 bp, Artificial sequence
  Single strand DNA oligonucleotide
aatcaaggatagagcgtgtggg

SEQ ID NO: 19 - DNA, 24 bp, Artificial sequence
  Single strand DNA oligonucleotide
gctgttttcttccctcttcgtatg

SEQ ID NO: 20 - DNA, 24 bp, Artificial sequence
  Single strand DNA oligonucleotide
cagtctcagtagcgcaaacaattt

SEQ ID NO: 21 - DNA, 21 bp, Artificial sequence
  Single strand DNA oligonucleotide
ttcagcaatggaaagaaatgg

SEQ ID NO: 22 - DNA, 20 bp, Artificial sequence
  Single strand DNA oligonucleotide
cccctgaaatgtttccaaga

SEQ ID NO: 23 - DNA, 20 bp, Artificial sequence
  Single strand DNA oligonucleotide
gaacgtgtgattggcagaaa

SEQ ID NO: 24 - DNA, 20 bp, Artificial sequence
  Single strand DNA oligonucleotide
tcgaaaacatggagttgcag

SEQ ID NO: 25 - DNA, 11190 bp, Homo sapiens
  misc_feature (861)..(861): Can be C or A
cagaggacaacatagataggttcggagactactcagagaaagggagagagaatgctaggttatctttcagctgttttagctgcttattgttccagctttagccaccatctgactgcaattgtatgagccagaaatagccaggtaaaattgtcttcccaaattcctagtgcacaaaaactgtcagaggctgggggcagtggctcactcctgtaatcccagcagcactttgggaagccagggtgggagaactgcttgagcccaggagttagaggcaacataacaacaactcatctctaatgaaaaaaaaaaaaaaagaaagaaaaaggccaggcacagtggctcacgcctgtaatcccagcactttaggaagccaaggagggtggatcatgaggtcaggagtttgagaccagcctggccaatatggtgaaaccccgtctctactaaaaacacaaaaatcagccacgcatagtggctggtgcctgtaatcacagctactcaggaggctaaggcaggagaatcacttgaatccgggaggcagaggttgcagtgagccgagattgcaccactgcatttcagcctgggcagcagagtgggactgtctcacattaattaattaattgattaaatgctagaagaaaatatggtcaaaatcttttataactcttggagtgaggaaggcaatctcggctcactacaacttccacctcccgggttcaaatgattctcctgcctcagcctcctgagtagctgggattacaggcacccaccaccatgcccggctaatttttgtatttttagtagagacgaggtttcgccatgttggccaagcttgtcttaaactcctgacctcaggtgatccanccacctcagcctcccaaattgctgggattacaggcatgagccaccatgccccgcctaattttaaaaatgtttataaaganagagtcttaccacgttgtccagacctgtctcaaactcctgggctcaagtcattgtcttgtctcagcctcccaaagttctgggactacaggtgtgcaccaccacacctggctaattttgtttattttttatagagtgagggtctccctgtgttgtccagggtgtctcaaactcctgggctcaagtggtcctcctgcctcagcctcccaaagtgctgggattataggcataagccactgcacctggccccagtgtgtgtcttctgaggctaagtcataaagagattgcggcttctattttgcttcctcttagatcactcattctgggggaaaccaacaataaataacaggaacacaccaggagatcagagaaagctgagatacaggcctaatgcccagtcctctgtattcatttctaattgctgctgtaacaagttactgcacatttaatgatgtaaaacaacacaaatttatcttacagttctgtagcttagaagtctgacagagtctcactgggctgaaatcaagatgttgatagttccttcccggtgactctagagtgagaatccagttctttaccctttcttgcctttagaggccacccgtatttattagctcacaatccccttcctccatcttcaaaccagaaacattgcagctctctgtgtcttttttctttactcacatctccctctgactttcttctgccatcctccttcattttttaaggacccctgtgggccaggcatggtggcttatgcctgtaatcccagcactttgagaggcggaggcaggcgggtcacctgaggtcaggagttccagaccagcctggccaacatggcaaaaccccatctccactgaaaatacaaaaattgggccaggcacggcggctcacacccgtaatccccatactttgagaggctgaggcaggtggatcacttgaggtcaggagttcaagaccagcctggccaacatggtgaaatcccgtctctactaaaaaaaaaaaaaattacaataattagtcaggcgtggtggccagcacctataatcccagctacttgggaggttgaggcacaagaattgcttgaacccgagagggggaggttgcagtgagctgagattgcaccaccgtactccagcctggatgacagagcaagactctgtctcaaaataaaaattaagataaataataaaaaataaataaaaatcagcaaattaggccaggtgtggtggtggctcatgcctgtactcccagcactttgaggtgggaaggttgcttgaaaccagaagtttagaccagcctgggaaacaaagtgagaccccatctctacaaaaataaaaataaattagccaggtgtggtgggacgtacttgtactcctagctactagggaggctgaggtgcaagaatcgcttgaacctgggagacggaggttgcagtgagttatgacagtgccactacactccagcctgggtgtcaagggtctttgaaaaaataaacaaaataggctgggcgtggtggcttacacttgtaatcccagcactttgggaggctgaggcaggcagatcacaaagtcaggagttcgagaccagcctggccaacacagtgaaaccccgtctctactaaaaatacaaaaattagccaggcatggttgcacgcgcctgtagtcccaactgctcgggaggctgaggcaggagaatggcttgaacctgggggggggcggaagttataatgagccaagattgtgccactgcactccagcctaggcaacagagcgagactccgtctcaaaaataaaaataaaaataaatcaatagagcctggtatgatggctcacgcctataatcccagcactttgggaggcccaggtgggtggatcatctgaggtcaggagtttgagaccagcctgaccaacatggagaaaccccatctctactaaaaatacaaaaaattagccaggcgtggtggcacatgcctataatcccagctactcaggagcctgaggcaggagaatcgctttaaccggggaggtggaggttgcggtgagtcgagatagcaccattgcattctagcctgggcaacaagagcaaaactccatctcaaaaataaataaataaatagatagataggctgggcacggtggctcacgcctgtaatcccagcactttgggaggccgaggtgggcggatcacctgaggtcaggagttcaagaccagcctggccaatatagcgaaaccctgtctctactaaaaatttaaaaaattagccaggcatgatggcgggtgcctgtaatcccagctactcgggaggctgaggcaggagaatctctcgaacctggcaggcggaggttgcagtgagccgagatcacaccattgcactccagttgggcaacaagagcgaaactccacctcaaaaacaaaataaaataaaataaaatcaggagattggtcagcttaattccatctgcaaccttaattcccttttggccatttaacctatcatattcacaagttccagggattcatgcagggacatctttggtgaccattattctgtctaccacactctctagaagagagtagatgtgagaaacagcatctggagagggaggagccagcaggagagggaaatatcacagacgccagaggaagagagttcccagaagggtaggtgcaacagtaagggatccctctgggaagtcaagcaagagaagacctgaaaaacaaccattggccnggtgcggtggctcacgcctataatcctagcattttgggaggccgaggtgggtggatcacttgaggtcaggagtttaagacaagcctggccaacatggtgaaaccctgtctctactaaaaatacaaaaattagccagacatggtggcaggcacctgtagtccaaactacttgggaggctgaggcacgagaaccacttgaaccctggaggcggaagttgcagtgagccgagatggcaccgctgcactccagcctgggcgacagagtgagactccgtctcaaaataagtaaataaataaataaataaccattgggtctagcgtcttggtgacaccagccactaggtgtcagggcaatgctggaggcagcagctagtttatggcgtgggcagcaaggaagaagtgaggaagtagagactgggagtgtagaatctttccaaaaacttgaatgtggaggggtagatgggataatgccgggagaggaggctggtgaaggacctcttagtgaagggagatttgttgctgtttctgagatgggaaagagaaacaagttgcagtgtagatggggaggatggagaggttgaaggtgtaggagagatgggtcaccatcagatgggacgtctgtgaaggagagacctcatctggcccacagcttggaaaggagagactgactgttgagttgatgcaagctcaggtgttgccaggcgggcgccatgatagtagagaggttaggatactgtcaagggtgtgtgtggccaaaggagtggttctgtgaatgtatgggagaaagggagaccgaccaccaggaagcactggtgaggcaggacccgggaggatgggaggctgcagcccgaatggtgcctgaaatagtttcaggggaaatgcttggttcccgaatcggatcgccgtattcgctggatcccctgatccgctggtctctaggtcccggatgctgcaattcttacaacaggacttggcatagggtaagcgcaaatgctgttaaccacactaacacacttttttttttcttttttttttttgagacagagtctcactctgtcggcctggctggagtgcagtggcacgatctcggctcactgcaacctccggctccccggctcaagcaattctcctgcctcagcctcccgagtagctgggattacaggcatgtgccaccacgcccggctaatttttgtatttttagttgagatggggtttcaccatgttggcgaggctggtcttgaactcctgacctcaggtaatccgccagcctcggcctcccaaagtgctgggattacaagcgtgagccaccgtgcccggccaacagtttttaaatctgtggagacttcatttcccttgatgccttgcagccgcgccgactacaactcccatcatgcctggcagccgctggggccgcgattccgcacgtcccttacccgcttcactagtcccggcattcttcgctgttttcctaactcgcccgcttgactagcgccctggaacagccatttgggtcgtggagtgcgagcacggccggccaatcgccgagtcagagggccaggaggggcgcggccattcgccgcccggcccctgctccgtggctggttttctccgcgggcgcctcgggcggaacctggagataatgggcagcacctgggggagccctggctgggtgcggctcgctctttgcctgacgggcttagtgctctcgctctacgcgctgcacgtgaaggcggcgcgcgcccggnaccgggattaccgcncgctctgngacgtgggcaccgccatcagctgttcgcgcgtcttctcctccaggtgtgcacgggagtgggaggcgtggggcctcggagcagggcggccaggatgccagatgattattctggagtctgggatcggtgtgcccggggaacggacacggggctggactgctcgcggggtcgttgcacaggggctgagctacccagcgatactggtgttcgaaataagagtgcgaggcaagggaccagacagtgctggggactgggattattccggggactcgcacgtgaattggatgccaaggaataacggtgaccaggaaaggcggggaggcaggatggcggtagagattgacgatggtctcaaggacggcgcgcaggngaaggggggtgttggcgatggctgcgcccaggaacaaggtggcccggtctggctgtgcgtgatggccaggcgttagcataatgacggaatacagaggaggcgagtgagtggccagggagctggagattctggggtccagggcaaagataatctgcccccgactcccagtctctgatgcaaaaccgagtgaaccgttataccagccttgccattttaagaattacttaagggccgggcgcggtggcccactcctgtaatcccagcactttgggaggccgaggcggatggatcacttgaagtcaggagttgaccagcctggccaacatggtgaaagcctgtctctaccaaaaatagaaaaattaatcgggcgctatggcgggtgccttaatcccagctactcgggggggctaaggcaggagaatcgcttgaacccgggaggcggaggtttcagtgagccgagatcgcgccactgcactccagcctgggccagagtgagactccgtctcaaaaaaaaaaaaaaaaaaaaaaaaaaagagacttacttaaggtctaagatgaaaagcagggcctacggagtagccacgtccgggcctggtctggggagaggggaggatagggtcagtgacatggaatcctgacgtggccaaaggtgcccggtgccaggagatcatcgacncttggactaggatgggaggtcggggaacagaggatagcccaggtggcttcttggaaatcacctttctcgggcagggtccaaggcactgggttgacagtcctaacctggttccaccccaccccacccctctgccaggtggggcaggggtttcgggctggtggagcatgtgctgggacaggacagcatcctcaatcaatccaacagcatattcggttgcatcttctacacactacagctattgttaggtgagtggctccgccccctccctgcccgccccgccccgcccctcatcccccttggtcagctcagccccactccatgcaatcttggtgatccacacagctgacagccagctagctgctcatcacngagcgtcctgcgggtggggatgtggggaggtaactaacaggagtcttttaattggtttaagtactgttagaggctgaagggcccttaaagacatcctaggtccccaggttttttgtttgttgttgttttgagacagggtctggctctgttgcccaaagtgaggtctaggatgcccttagtgtgcactggcgtgatctcagttcatggcaacctctgcctccctgcccaagggatcctcccaccttagcctcccaagcagctggaatcacaggcgtgcaccactatgcccagctaatttttgtttttgtttttttttggtagagatggtgtctcgccatgttgcccaggctggtctcaagcaatctgtctgcctcagcctcccaaagtgctggggggattacaggcgtgagctaccatgccccaccaacaccccagttttgtggaaaagatgccgaaattcctttttaaggagaagctgagcatgagctatcttttgtctcatttagtgctcagcaggaaaatttgtatctagtcccataagaacagagagaggaaccaagggagtggaagacgatggcgccccaggccttgctgatgccatatgccggagatgagactatccattaccacccttcccagcaggctcccacgctccctttgagtcacccttcccagctccagagaaggcatcactgagggaggcccagcaccacggtcctggctgacacatggttcagacttggccgatttatttaagaaattttattgctcagaactttccctccctgggcaatggcaagagcttcagagaccagtcccttggaggggacctgttgaagccttctttttttttttttttaagaaataatcttgctctgttgcccaggctggagtgcagtggcacaatcatagctcactgtaacctggctcaagcgatcctcctgagtagctaggactataggcatgtcactgcacccagctaattttttttttttttttttttttttttttgcgacatagtctcgctctgtcaccaggctggagtgcagtggcacgatcttggctcactgcaacctctgcctcccgggttcaagcaattttcctgcctcagcctcctgagtagctgggactacaggcgcgtgtcaccacgcccagctaatttttgtatttttagtggagacagggtttcaccatgttggctaggatggtctcaatctcttgacctggtgatccatccgccttggcctcccaaagtgctaggattacaggcgtgagtcaacctcaccgggcattttttttttgagacgaagtcttgctcttgctgcccaagctggaatgtggtggcatgatctcggctcactgcaacctccacctcctaggttcaagcgattctccaccttagcctccccagcagctgggattacaggtgcccatcaacacacccggctaatttttgtatttttattagagatggggttttgccatgttggccaggctgctctcgaactcctaacctcaggtgatccacccccattggcctcccaaaatactgggattacaggcatgagccaccgtgcccagctgaatttctaaatttttgatagagatcgggtctttctatgttgcccaagctggtcttgaactcctagcctaaagcagtcttcccacctcggcctcccagagtgtttggaatacgtgcgtaagccaccacatctgccctggagcctcttgttttagagacccttcccagcagctcctggcatctaggtagtgcagtgacatcatggagtgttcgggaggtggccagtgcctgaagcccacaccggaccctcttctgccttgcaggttgcctgcggacacgctgggcctctgtcctgatgctgctgagctccctggtgtctctcgctggttctgtctacctggcctggatcctgttcttcgtgctctatgatttctgcattgtttgtatcaccacctatgctatcaacgtgagcctgatgtggctcagtttccggaaggtccaagaaccccagggcaaggctaagaggcactgagccctcaacccaagccaggctgacctcatctgctttgctttggcatgtgagccttgcctaagggggcatatctgggtccctagaaggccctagatgtggggcttctagattaccccctcctcctgccatacccncacatgacaatggaccaaatgtgccacacgctcgctcttttttacacccagtgcctctgactctgtccccatgggctggtctccaaagctctttccattgcccagggagggaaggttctgagcaataaagtttcttagatcaatcagccaagtctgaaccatgtgtctgccatggactgtggtgctgggcctccctcggtgttgccttctctggagctgggaagggtgagtcagagggagagtggagggcctgctgggaagggtggttatgggtagtctcatctccagtgtgtggagtcagcaaggcctggggcaccattggcccccacccccaggaaacaggctggcagctcgctcctgctgcccacaggagccaggcctcctctcctgggaaggctgagcacacacctggaagggcaggctgcccttctggttctgtaaatgcttgctgggaagttcttccttgagtttaactttaacccctccagttgccttatcgaccattccaagccagtattggtagccttggagggtcagggccaggttgtgaaggtttttgttttgcctattatgccctgaccacttacctacatgccaagcactgtttaagaacttgtgttggcagggtgcagtggctcacacctgtaatccctgtactttgggaggccaaggcaggaggatcacttgaggccaggagttccagaccagcctgggcaaaatagtgagacccctgtctctacaaaaaaaaaaaaaaaaaaaaattagccaggcatggtggtgtatgtacctatagtcccaactaatcgggaagctggcgggaagactgcttgagcccagaaggttgaggctgcagtgagccatgatcactgcactccagcctgagcaacagagcaagaccgtctccaaaaaaaaacaaaaaacaaaaaaaaacttgtgttaacgtgttaaactcgtttaatctttacagtgatttatgaggtgggtactattattatccctatcttgatgatagggacagagtggctaattagtatgcctgagatcacacagctactgcaggaggctctcaggatttgaatccacctggtccatctggctccagcatctatatgcttttttttttgttggtttgtttttgagacggactttcgctcttgttgtccaggctggagtgcaatggcacaatttcggctcaccacaacctccacctcccaggttcaagtgattctcctgcctcagcctcccgagtagctgggattacaggcatgcgccaccaggcctggctaattttgtatttttagtagagacagtgtttctctatgttggtcaggctggtctcgcactcccgacctcaggtgatctgcctgctttggcctcccaaagtgctgggattacaggcgtgagccaccatgcccggccagcatctataatatatgctaaaccttacccacagctgcctctgtcttggggtcagggttcttgaatggtctgggccagcctgagttaggagtgggaagaggaagaggaggcacgcgccaaagtgttcttcccgcctgaccgagtcctctgcgcctatgaggaaggtgggcaccgctgtcaccctgtttcccaggaagccagcaggcccagggagatactgggatttgccaaaggctcacagcgagcaagcagcagtgggtgctggctcctcaccactcagcctggcacacactgtctctgagcagacctgggagaaggcatgtgaaacccacctagccctggctggaagagaagtgggggagtggaaatgaaaagtggggactgggaggagctgcccacgtgccctccatggttccaggatggcactttcatgctgggagtgagcttgccctgtgccaggctgtcattgagacgttttccattcaacaactgtttctcagacaaggcacaggcaggttagctctcccaggccactcaagtagtaaggaaacagatttaaacccagacaatttggctccacggctaaggcccagagaggggtggtgacaagcctagggtcacacagccacagccccagagctaggaactgctggctagggcaagcagacagctgaatcctagccaactggaggtggagcagaggtggagccagggagacccaggtggcctc

SEQ ID NO: 26 - DNA, 3320 bp, Homo sapiens
  misc_feature (73)..(73): Can be G or A
gtgggtgagcggcggccacggcatcctgtgctgtgggggctacgaggaaagatctaattatcatggacctgcnacagtttcttatgtgcctgtccctgtgcacagcctttgccttgagcaaacccacagaaaagaaggaccgtgtacatcatgagcctcagctcagtgacaaggttcacaatgatgctcagagttttgattatgaccatgatgccttcttgggtgctgaagaagcaaagacctttgatcagctgacaccagaagagagcaaggaaaggcttggaaagattgtaagnaaaatagatggcgacaaggacgggtttgtcactgtggatgagctcaaagactggattaaatttgcacaaaagcgctggatttacgaggatgtagagcgacagtggaaggggcatgacctcaatgaggacggcctcgtttcctgggaggagtataaaaatgccacctacggctacgttttagatgatccagatcctgatgatggatttaactataaacagatgatggttagagatgagcggaggtttaaaatggcagacaaggatggagacctcattgccaccaaggaggagttcacagctttcctgcaccctgaggagtatgactacatgaaagatatagtagtacaggaaacaatggaagatatagataagaatgctgatggtttcattgatctagaagagtatattggtgacatgtacagccatgatgggaatactgatgagccagaatgggtaaagacagagcgagagcagtttgttgagtttcgggataagaaccgtgatgggaagatggacaaggaagagaccaaagactggatccttccctcagactatgatcatgcagaggcagaagccaggcacctggtctatgaatcagaccaaaacaaggatggcaagcttaccaaggaggagatcgttgacaagtatgacttatttgttggcagccaggccacagattttggggaggccttagtacggcatgatgagttctgagctgcggaggaaccctcatttcctcaaaagtaatttatttttacagcttctggtttcacatgaaattgtttgcgctactgagactgttactacaaactttttangacatgaaaaggcgtaatgaaaaccatcccgtccccattcctcctcctctctgagggactggagggaagccgtgcttctgaggaacaactctaattagtacacttgtgtttgtagatttacactttgtattatgtattaacatggcgtgtttatttttgtatttttctctggttgggagtatgatatgaaggatcaagatcctccactcacacatgtagacaaacattagctctttactctttctcaaccccttatatgattttaataattctcacttcactaattttgtaagcctgagatcaataagaaatgttcaggagagaggaaagaaaaaatatatatgctccacaatttatatttagagagagaacacttagtcttgcctgtcaaaaagtccaacatttcataggtagtaggggccacatattacattcagttgctataggtccagcaactgaacctgccattacctgggcaaggaaagatccctttgctctaggaaagcttggcccaaattgattttcttctttttccccctgtaggactgactgttggctaattttgtcaagcacagctgtggtgggaagagttagggccagtgtcttgaaaatcaatcaagtagtgaatgtgatctctttgcagagctatagatagaaacagctggaaaactaaaggaaaaatacaaatgttttcggggcatacattttttttctgggtgtgcatctgttgaaatgctcaagacttaattatttgccttttgaaatcactgtaaatgcccccatccggttcctcttcttcccaggtgtgccaaggaattaatcttggtttcactacaattaaaattcactcctttccaatcatgtcattgaaagtgcctttaacgaaagaaatggtcactgaatgggaattctcttaagaaaccctgagattaaaaaaagactatttggataacttataggaaagcctagaacctcccagtagagtggggatttttttcttcttccctttctcttttggacaatagttaaattagcagtattagttatgagtttggttgcagtgttcttatcttgtgggctgatttccaaaaaccacatgctgctgaatttaccagggatcctcatacctcacaatgcaaaccacttactaccaggcctttttctgtgtccactggagagcttgagctcacactcaaagatcagaggacctacagagagggctctttggtttgaggaccatggcttacctttcctgcctttgacccatcacaccccatttcctcctctttccctctccccgctgccaaaaaaaaaaaaaaggaaacgtttatcatgaatcaacagggtttcagtccttatcaaagagagatgtggaaagagctaaagaaaccaccctttgttcccaactccactttacccatattttatgcaacacaaacactgtccttttgggtccctttcttacagatggacctcttgagaagaattatcgtattccacgtttttagccctcaggttaccaagataaatatatgtatatataacctttattattgctatatctttgtggataatacattcaggtggtgctgggtgatttattataatctgaacctaggtatatcctttggtcttccacagtcatgttgaggtgggctccctggtatggtaaaaagccaggtataatgtaacttcaccccagcctttgtactaagctcttgatagtggatatactcttttaagtttagccccaatatagggtaatggaaatttcctgccctctgggttccccatttttactattaagaagaccagtgataatttaataatgccaccaactctggcttagttaagtgagagtgtgaactgtgtggcaagagagcctcacacctcactaggtgcagagagcccaggccttatgttaaaatcatgcacttgaaaagcaaaccttaatctgcaaagacagcagcaagcattatacggtcatcttgaatgatccctttgaaattttttttttgtttgtttgtttaaatcaagcctgaggctggtgaacagtagctacacacccatattgtgtgttctgtgaatgctagctctcttgaatttggatattggttattttttatagagtgtaaaccaagttttatattctgcaatgcgaacaggtacctatctgtttctaaataaaactgtttacattcaaaaa

SEQ ID NO: 27 - DNA, 1835 bp, Homo sapiens
  misc_feature (430)..(430): Can be C or T
atggattctcttgtggtccttgtgctctgtctctcatgtttgcttctcctttcactctggagacagagctctgggagaggaaaactccctcctggccccactcctctcccagtgattggaaatatcctacagataggtattaaggacatcagcaaatccttaaccaatctctcaaaggtctatggcccggtgttcactctgtattttggcctgaaacccatagtggtgctgcatggatatgaagcagtgaaggaagccctgattgatcttggagaggagttttctggaagaggcattttcccactggctgaaagagctaacagaggatttggaattgttttcagcaatggaaagaaatggaaggagatccggcgtttctccctcatgacgctgcggaattttgggatggggaagaggagcattgaggacngtgttcaagaggaagcccgctgccttgtggaggagttgagaaaaaccaaggcctcaccctgtgatcccactttcatcctgggctgtgctccctgcaatgtgatctgctccattattttccataaacgttttgattataaagatcagcaatttcttaacttaatggaaaagttgaatgaaaacatcaagattttgagcagcccctggatccagatctgcaataatttttctcctatcattgattacttcccgggaactcacaacaaattacttaaaaacgttgcttttatgaaaagttatattttggaaaaagtaaaagaacaccaagaatcaatggacatgaacaaccctcaggactttattgattgcttcctgatgaaaatggagaaggaaaagcacaaccaaccatctgaatttactattgaaagcttggaaaacactgcagttgacttgtttggagctgggacagagacgacaagcacaaccctgagatatgctctccttctcctgctgaagcacccagaggtcacagctaaagtccaggaagagattgaacgtgtgattggcagaaaccggagcccctgcatgcaagacaggagccacatgccctacacagatgctgtggtgcacgaggtccagagatacnttgaccttctccccaccagcctgccccatgcagtgacctgtgacattaaattcagaaactatctcattcccaagggcacaaccatattaatttccctgacttctgtgctacatgacaacaaagaatttcccaacccagagatgtttgaccctcatcactttctggatgaaggtggcaattttaagaaaagtaaatacttcatgcctttctcagcaggaaaacggatttgtgtgggagaagccctggccggcatggagctgtttttattcctgacctccattttacagaactttaacctgaaatctctggttgacccaaagaaccttgacaccactccagttgtcaatggatttgcctctgtgccgcccttctaccagctgtgcttcattcctgtctgaagaagagcagatggcctggctgctgctgtgcagtccctgcagctctctttcctctggggcattatccatctttgcactatctgtaatgccttttctcacctgtcatctcacattttcccttccctgaagatctagtgaacattcgacctccattacggagagtttcctatgtttcactgtgcaaatatatctgctattctccatactctgtaacagttgcattgactgtcacataatgctcatacttatctaatgtagagtattaatatgttattattaaatagagaaatatgatttgtgtattataattcaaaggcatttcttttctgcatgatctaaataaaaagcattattatttgctg

SEQ ID NO: 28 - protein, 163 aa, Homo sapiens
  misc_feature (36)..(36): Can be D or Y
MGSTWGSPGWVRLALCLTGLVLSLYALHVKAARARXRDYRXLCDVGTAISCSRVFSSRWGRGFGLVEHVLGQDSILNQSNSIFGCIFYTLQLLLGCLRTRWASVLMLLSSLVSLAGSVYLAWILFFVLYDFCIVCITTYAINVSLMWLSFRKVQEPQGKAKRH

SEQ ID NO: 29 - protein, 315 aa, Homo sapiens
  misc_feature (4)..(4): Can be R or Q
MDLXQFLMCLSLCTAFALSKPTEKKDRVHHEPQLSDKVHNDAQSFDYDHDAFLGAEEAKTFDQLTPEESKERLGKIVXKIDGDKDGFVTVDELKDWIKFAQKRWIYEDVERQWKGHDLNEDGLVSWEEYKNATYGYVLDDPDPDDGFNYKQMMVRDERRFKMADKDGDLIATKEEFTAFLHPEEYDYMKDIVVQETMEDIDKNADGFIDLEEYIGDMYSHDGNTDEPEWVKTEREQFVEFRDKNRDGKMDKEETKDWILPSDYDHAEAEARHLVYESDQNKDGKLTKEEIVDKYDLFVGSQATDFGEALVRHDEF

SEQ ID NO: 30 - protein, 490 aa, Homo sapiens
  misc_feature (144)..(144): Can be C or R
MDSLVVLVLCLSCLLLLSLWRQSSGRGKLPPGPTPLPVIGNILQIGIKDISKSLTNLSKVYGPVFTLYFGLKPIVVLHGYEAVKEALIDLGEEFSGRGIFPLAERANRGFGIVFSNGKKWKEIRRFSLMTLRNFGMGKRSIEDXVQEEARCLVEELRKTKASPCDPTFILGCAPCNVICSIIFHKRFDYKDQQFLNLMEKLNENIKILSSPWIQICNNFSPIIDYFPGTHNKLLKNVAFMKSYILEKVKEHQESMDMNNPQDFIDCFLMKMEKEKHNQPSEFTIESLENTAVDLFGAGTETTSTTLRYALLLLLKHPEVTAKVQEEIERVIGRNRSPCMQDRSHMPYTDAVVHEVQRYXDLLPTSLPHAVTCDIKFRNYLIPKGTTILISLTSVLHDNKEFPNPEMFDPHHFLDEGGNFKKSKYFMPFSAGKRICVGEALAGMELFLFLTSILQNFNLKSLVDPKNLDTTPVVNGFASVPPFYQLCFIPV

SEQ ID NO: 31 - DNA, 16 bp, Artificial sequence
  Single strand DNA oligonucleotide
gggaccgggattaccg

SEQ ID NO: 32 - DNA, 20 bp, Artificial sequence
  Single strand DNA oligonucleotide
gctgcttccactatggcttc

SEQ ID NO: 33 - DNA, 21 bp, Artificial sequence
  Single strand DNA oligonucleotide
ttgggttctgaatctctccaa

SEQ ID NO: 34 - DNA, 1856 bp, Homo sapiens
  misc_feature (612)..(612): Can be T or C
ctgacttggcaggactgtgcaattgtcagaaggccgtggggagtgggggccagtgcctgcagcctgccctgcctctctcacaggcccttagagcatcgccaggtgcagagctccacagctctctttcccaaggagtaatcagagggtgagaacgtggagcctggtggacaggtgaaagcactgggatctttctgcccagaaaggggaaagttgcacatttatatcctagagggaagcgacagcagtgcttctccctgtgctgaggtacaggagccatgtggctagaaatcctcctcacttcagtgctgggctttgccatctactggttcatctcccgggacaaagaggaaactttgccacttgaagatgggtggtgggggccaggcacgaggtccgcagccagggaggacgacagcatccgccctttcaaggtggaaacgtcagatgaggagatccacgacttacaccagaggatcgataagttccgtttcaccccacctttggaggacagctgcttccactatggcttcaactccaactacctgaagaaagtcatctcctactggcggaatgaatttgactggaagaagcaggtggagattctcaacaganaccctcacttcaagactaagattgaagggctggacatccacttcatccacgtgaagcccccccagctgcccgcaggccataccccgaagcccttgctgatggtgcacggctggcccggctctttctacgagttttataagatcatcccactcctgactgaccccaagaaccatggcctgagcgatgagcacgtttttgaagtcatctgcccttccatccctggctatggcttctcagaggcatcctccaagaaggggttcaactcggtggccaccgccaggatcttttacaagctgatgctgcggctgggcttccaggaattctacattcaaggaggggactgggggtccctgatctgcactaatatggcccagctggtgcccagccacgtgaaaggcctgcacttgaacatggctttggttttaagcaacttctctaccctgaccctcctcctgggacagcgtttcgggaggtttcttggcctcactgagagggatgtggagctgctgtaccccgtcaaggagaaggtattctacagcctgatgagggagagcggctacatgcacatccagtgcaccaagcctgacaccgtaggctctgctctgaatgactctcctgtgggtctggctgcctatattctagagaagttttccacctggaccaatacggaattccgatacctggaggatggaggcctggaaaggaagttctccctggacgacctgctgaccaacgtcatgctctactggacaacaggcaccatcatctcctcccagcgcttctacaaggagaacctgggacagggctggatgacccagaagcatgagcggatgaaggtctatgtgcccactggcttctctgccttcccttttgagctattgcacacgcctgaaaagtgggtgaggttcaagtacccaaagctcatctcctattcctacatggttcgtgggggccactttgcggcctttgaggagccggagctgctcgcccaggacatccgcaagttcctgtcggtgctggagcggcaatgacccacccctctccccccgcctgccacctccccccacaagtgccctccaggcttttcttggggaagataccccttttctgaggaatgagtttgcctccgtcccctgcccatgctgggagcccacgctcaccccctcacccctccaagctcactccccaacccccaactccgtgtggtaagcaacatggctttgatgataaacgactttactcta

SEQ ID NO: 35 - protein, 455 aa, Homo sapiens
  misc_feature (113)..(113): Can be Y or H
MWLEILLTSVLGFAIYWFISRDKEETLPLEDGWWGPGTRSAAREDDSIRPFKVETSDEEIHDLHQRIDKFRFTPPLEDSCFHYGFNSNYLKKVISYWRNEFDWKKQVEILNRXPHFKTKIEGLDIHFIHVKPPQLPAGHTPKPLLMVHGWPGSFYEFYKIIPLLTDPKNHGLSDEHVFEVICPSIPGYGFSEASSKKGFNSVATARIFYKLMLRLGFQEFYIQGGDWGSLICTNMAQLVPSHVKGLHLNMALVLSNFSTLTLLLGQRFGRFLGLTERDVELLYPVKEKVFYSLMRESGYMHIQCTKPDTVGSALNDSPVGLAAYILEKFSTWTNTEFRYLEDGGLERKFSLDDLLTNVMLYWTTGTIISSQRFYKENLGQGWMTQKHERMKVYVPTGFSAFPFELLHTPEKWVRFKYPKLISYSYMVRGGHFAAFEEPELLAQDIRKFLSVLERQ

SEQ ID NO: 36 - DNA, 16 bp, Artificial sequence
  Single strand DNA oligonucleotide
ggtaccgggattaccg

SEQ ID NO: 37 - DNA, 780 bp, Homo sapiens
  misc_feature (514)..(514): Can be G or C
ctgagcactaaatgagacaaaagatagctcatgctcagcttctccttaaaaaggaatttcggcatcttttccacaaaactggggtgttggtggggcatggtagctcacgcctgtaatccccccagcactttgggaggctgaggcagacagattgcttgagaccagcctgggcaacatggcgagacaccatctctaccaaaaaaaaacaaaaacaaaaattagctgggcatagtggtgcacgcctgtgattccagctgcttgggaggctaaggtgggaggatcccttgggcagggaggcagaggttgccatgaactgagatcacgccagtgcacactaagggcatcctagacctcactttgggcaacagagccagaccctgtctcaaaacaacaacaaacaaaaaacctggggacctaggatgtctttaagggcccttcagcctctaacagtacttaaaccaattaaaagactcctgttagttacctccccacatccccacccgcaggacgctcngtgatgagcagctagctggctgtcagctgtgtggatcaccaagattgcatggagtggggctgagctgaccaagggggatgaggggcggggcggggcgggcagggagggggcggagccactcacctaacaatagctgtagtgtgtagaagatgcaaccgaatatgctgttggattgattgaggatgctgtcctgtcccagcacatgctccaccagcccgaaacccctgccccacctggcagaggggtggggtggggtggaaccaggt

SEQ ID NO: 38 - DNA, 511 bp, Homo sapiens
  misc_feature (256)..(256): Can be A or G
gctcactgaaacctccgcctcccgggttcaagcgattctcctgccttagcccccccgagtagctgggattaaggcacccgccatagcgcccgattaatttttctatttttggtagagacaggctttcaccatgttggccaggctggtcaactcctgacttcaagtgatccatccgcctcggcctcccaaagtgctgggattacaggagtgggccaccgcgcccggcccttaagtaattcttaaaatggcaaggctngtataacggttcactcggttttgcatcagagactgggagtcgggggcagattatctttgccctggaccccagaatctccagctccctggccactcactcgcctcctctgtattccgtcattatgctaacgcctggccatcacgcacagccagaccgggccaccttgttcctgggcgcagccatcgccaacaccccccttcacctgcgcgccgtccttgagaccatcgtcaatctctaccgccatcctgcctcccc

SEQ ID NO: 39 - DNA, 117 bp, Homo sapiens
  misc_feature (57)..(57): Can be C or T
gtttctccctcatgacgctgcggaattttgggatggggaagaggagcattgaggacngtgttcaagaggaagcccgctgccttgtggaggagttgagaaaaaccaagggtgggtgac

SEQ ID NO: 40 - DNA, 116 bp, Homo sapiens
  misc_feature (56)..(56): Can be A or C
agacaggagccacatgccctacacagatgctgtggtgcacgaggtccagagatacnttgaccttctccccaccagcctgccccatgcagtgacctgtgacattaaattcagaaact

SEQ ID NO: 41 - DNA, 121 bp, Homo sapiens
  misc_feature (61)..(61): Can be A or G
tgatttccaagaagccacctgggctatcctctgttccccgacctcccatcctagtccaagngtcgatgatctcctggcaccgggcacctttggccacgtcaggattccatgtcactgaccc

SEQ ID NO: 42 - DNA, 121 bp, Homo sapiens
  misc_feature (61)..(61): Can be A or G
gggtccctagaaggccctagatgtggggcttctagattaccccctcctcctgccatacccncacatgacaatggaccaaatgtgccacacgctcgctcttttttacacccagtgcctctga

SEQ ID NO: 43 - DNA, 116 bp, Homo sapiens
  misc_feature (56)..(56): Can be A or G
tacaggagtgggccaccgcgcccggcccttaagtaattcttaaaatggcaaggctngtataacggttcactcggttttgcatcagagactgggagtcgggggcagattatctttgc

SEQ ID NO: 44 - DNA, 120 bp, Homo sapiens
  misc_feature (57)..(57): Can be G or T
tgacgggcttagtgctctcgctctacgcgctgcacgtgaaggcggcgcgcgcccggnaccgggattaccgcgcgctctgcgacgtgggcaccgccatcagctgttcgcgcgtcttctcct

SEQ ID NO: 45 - DNA, 121 bp, Homo sapiens
  misc_feature (61)..(61): Can be C or T
ttttctgtgggtttgctcaaggcaaaggctgtgcacagggacaggcacataagaaactgtngcaggtccatgataattagatcttgaagaaaatgaaaagcagttaattcaggaggcagta

SEQ ID NO: 46 - DNA, 120 bp, Homo sapiens
  misc_feature (58)..(58): Can be T or G
tagatggcgacaaggacgggtttgtcactgtggatgagctcaaagactggattaaatntgcacaaaagcgctggatttacgaggatgtagagcgacagtggaaggggcatgacctcaatg

SEQ ID NO: 47 - DNA, 113 bp, Homo sapiens
  misc_feature (61)..(61): Can be A or G
gttggcagccaggccacagattttggggaggccttagtacggcatgatgagttctgagctncggaggaaccctcatttcctcaaaagtaatttatttttacagcttctggttt

SEQ ID NO: 48 - DNA, 121 bp, Homo sapiens
  misc_feature (61)..(61): Can be C or T
gtcatctcctactggcggaatgaatttgactggaagaagcaggtggagattctcaacaganaccctcacttcaagactaagattgaaggtatgtttgcaaaacgccagccagagagggatg

SEQ ID NO: 49 - DNA, 120 bp, Homo sapiens
  misc_feature (62)..(62): Can be G or A
gccagcagccctctcttctgctcccctgagtggcctcggaagctggtgtcctactgcccccnaaggttgcaacaactgttgcccctcaaggcagcccctcagcccagtgtttcctgtgtg

SEQ ID NO: 50 - DNA, 30 bp, Artificial sequence
  Single strand DNA oligonucleotide
acgttggatgatgacgctgcggaattttgg

SEQ ID NO: 51 - DNA, 30 bp, Artificial sequence
  Single strand DNA oligonucleotide
acgttggatgcccagtctctgatgcaaaac

SEQ ID NO: 52 - DNA, 30 bp, Artificial sequence
  Single strand DNA oligonucleotide
acgttggatgttgactggaagaagcaggtg

SEQ ID NO: 53 - DNA, 30 bp, Artificial sequence
  Single strand DNA oligonucleotide
acgttggatgtgtcacaggtcactgcatgg

SEQ ID NO: 54 - DNA, 30 bp, Artificial sequence
  Single strand DNA oligonucleotide
acgttggatgtacttttgaggaaatgaggg

SEQ ID NO: 55 - DNA, 30 bp, Artificial sequence
  Single strand DNA oligonucleotide
acgttggatgtgacatggaatcctgacgtg

SEQ ID NO: 56 - DNA, 30 bp, Artificial sequence
  Single strand DNA oligonucleotide
acgttggatgaaaaaagagcgagcgtgtgg

SEQ ID NO: 57 - DNA, 30 bp, Artificial sequence
  Single strand DNA oligonucleotide
acgttggatgtacacacaggaaacactggg

SEQ ID NO: 58 - DNA, 30 bp, Artificial sequence
  Single strand DNA oligonucleotide
acgttggatgcactgtggatgagctcaaag

SEQ ID NO: 59 - DNA, 30 bp, Artificial sequence
  Single strand DNA oligonucleotide
acgttggatgctgcctcctgaattaactgc

SEQ ID NO: 60 - DNA, 30 bp, Artificial sequence
  Single strand DNA oligonucleotide
acgttggatgaagacgcgcgaacagctgat

SEQ ID NO: 61 - DNA, 30 bp, Artificial sequence
  Single strand DNA oligonucleotide
acgttggatgtcaactcctccacaaggcag

SEQ ID NO: 62 - DNA, 30 bp, Artificial sequence
  Single strand DNA oligonucleotide
acgttggatggcccggcccttaagtaattc

SEQ ID NO: 63 - DNA, 30 bp, Artificial sequence
  Single strand DNA oligonucleotide
acgttggatgtggcgttttgcaaacatacc

SEQ ID NO: 64 - DNA, 30 bp, Artificial sequence
  Single strand DNA oligonucleotide
acgttggatgctacacagatgctgtggtgc

SEQ ID NO: 65 - DNA, 30 bp, Artificial sequence
  Single strand DNA oligonucleotide
acgttggatgcagccaggccacagattttg

SEQ ID NO: 66 - DNA, 30 bp, Artificial sequence
  Single strand DNA oligonucleotide
acgttggatgacctgggctatcctctgttc

SEQ ID NO: 67 - DNA, 30 bp, Artificial sequence
  Single strand DNA oligonucleotide
acgttggatgttctagattaccccctcctc

SEQ ID NO: 68 - DNA, 30 bp, Artificial sequence
  Single strand DNA oligonucleotide
acgttggatgtcggaagctggtgtcctact

SEQ ID NO: 69 - DNA, 30 bp, Artificial sequence
  Single strand DNA oligonucleotide
acgttggatgttccactgtcgctctacatc

SEQ ID NO: 70 - DNA, 29 bp, Artificial sequence
  Single strand DNA oligonucleotide
acgttggatgacagggacaggcacataag

SEQ ID NO: 71 - DNA, 29 bp, Artificial sequence
  Single strand DNA oligonucleotide
acgttggatgtctacgcgctgcacgtgaa

SEQ ID NO: 72 - DNA, 17 bp, Artificial sequence
  Single strand DNA oligonucleotide
agaggagcattgaggac

SEQ ID NO: 73 - DNA, 18 bp, Artificial sequence
  Single strand DNA oligonucleotide
ccgagtgaaccgttatac

SEQ ID NO: 74 - DNA, 19 bp, Artificial sequence
  Single strand DNA oligonucleotide
ggtggagattctcaacaga

SEQ ID NO: 75 - DNA, 20 bp, Artificial sequence
  Single strand DNA oligonucleotide
gctggtggggagaaggtcaa

SEQ ID NO: 76 - DNA, 21 bp, Artificial sequence
  Single strand DNA oligonucleotide
gaggaaatgagggttcctccg

SEQ ID NO: 77 - DNA, 23 bp, Artificial sequence
  Single strand DNA oligonucleotide
cccggtgccaggagatcatcgac

SEQ ID NO: 78 - DNA, 17 bp, Artificial sequence
  Single strand DNA oligonucleotide
tggtccattgtcatgtg

SEQ ID NO: 79 - DNA, 19 bp, Artificial sequence
  Single strand DNA oligonucleotide
caacagttgttgcaacctt

SEQ ID NO: 80 - DNA, 20 bp, Artificial sequence
  Single strand DNA oligonucleotide
gctcaaagactggattaaat

SEQ ID NO: 81 - DNA, 21 bp, Artificial sequence
  Single strand DNA oligonucleotide
atctaattatcatggacctgc

SEQ ID NO: 82 - DNA, 22 bp, Artificial sequence
  Single strand DNA oligonucleotide
cagagcgcgcggtaatcccggt

(19) United States
(12) Patent Application Publication
(10) Pub. No.: US 2009/0226909 A1
(43) Pub. Date: Sep. 10, 2009

(54) Methods and kits for Determining Predisposition to Warfarin Resistance

(75) Inventors: Eva Gak, Tel-Aviv (IL); Hillel Halkin, Savyon (IL)

Correspondence Address: MARTIN D. MOYNIHAN d/b/a PRTSI, INC., P.O. BOX 16446, ARLINGTON, VA, 22215, US

(73) Assignee: Tel HaShomer Medical Research Infrastructure and Services Ltd., Ramat-Gan (IL)

(21) Appl. No.: 12/225,656

(22) PCT Filed: Mar. 28, 2007
(86) PCT No.: PCT/IL2007/000405

§ 371 Date: Apr. 2, 2009

Related U.S. Application Data
(60) Provisional application No. 60/786,350, filed on Mar. 28, 2006.
(60) Provisional application No. 60/834,559, filed on Aug. 1, 2006.

(51) Int. Cl. C12Q 1/68

(52) U.S. Cl. 435/6

(57) ABSTRACT

The present invention is of the high association of the 5417T allele of the VKORC1 gene with high coumarin dose requirements and which can be used to determine the predisposition of an individual to coumarin resistance. Specifically, the present invention provides methods and kits for determining the predisposition of an individual to coumarin resistance and for predicting the responsiveness of an individual to coumarin treatment.

### FIELD AND BACKGROUND OF THE INVENTION

The present invention relates methods and kits for determining predisposition to coumarin (e.g., warfarin) resistance and to methods and kits for predicting the responsiveness of an individual to coumarin treatment.

Coumarin derivatives such as warfarin, phenprocoumon and acenocoumarol (AC) are the most common anticoagulants prescribed worldwide for the prevention of thromboembolism. However, due to an extensive variability in individual response to coumarin derivatives (e.g., about 20-fold variability in the response to warfarin), their use requires careful clinical management to balance the risks of over-anticoagulation and bleeding with those of under-anticoagulation and blood clotting. As this relationship is multifactorial, coumarin therapy is individualized by carefully monitoring the anticoagulation status using the International Normalized Ratio (INR) as a standard of optimal relationship between the antithrombotic efficacy and the bleeding risk, particularly during the initial period of the treatment.

Factors affecting coumarin dose requirement include age, dietary vitamin K intake, certain diseases (e.g., heart failure or severe liver disease), medications (e.g., procor or aspirin) and ethnic origin (e.g., Chinese and Japanese require lower doses of warfarin as compared to Caucasians and African Americans). In addition, variations in the pharmacokinetics of the active S-enantiomer form of warfarin (S-warfarin), which is predominantly regulated by the cytochrome P450 CYP2C9 metabolizing enzyme, or the pharmacodynamic of warfarin, which limits the regeneration of reduced vitamin K that is essential for the production of active clotting factors, may also affect coumarin dose requirements. Thus, it has been demonstrated that genetic variants of the CYP2C9 gene (e.g., the CYP2C9*1, *2 and *3 variants) are associated with variations in the levels of enzymatic activity, particularly, the CYP2C9*3 variant was found to be a major contributor to warfarin sensitivity and the CYP2C9*1 variant (also referred to as CYP2C9 “wild type” allele) was found to be associated with higher warfarin doses of 28-39 mg/week (Higashi M K et al 2002; Sconce et al., 2005). In addition, ethnic stratification of CYP2C9*2 and *3 corresponds to the epidemiological studies of warfarin dose response in various populations.

To study the involvement of pharmacodynamic factors in coumarin sensitivity, mutation screenings were conducted in a candidate warfarin target, the vitamin K epoxide reductase (VKOR) enzyme, of which subunit 1 (the major component) is encoded by the VKORC1 gene (GenBank Accession No. AY587020).

Rare mutations in the VKORC1 gene have been associated with warfarin resistance. Rost S., et al. (2004) identified a rare VKORC1 mutation (R98W) that leads to familial defective vitamin K-dependent clotting factors (OMIM No. 607473), a condition characterized by bleeding tendency that is usually reversed by oral administration of vitamin K. Other rare mutations in the VKORC1 gene (V29L, V45A, R58G and L128R) were found in subjects with autosomal dominant warfarin resistance (OMIM No. 122700). Similarly, Harrington D J, et al. (2005) identified a VKORC1 missense mutation (V66M) in a warfarin resistant subject who required more than 25 mg of warfarin daily (i.e., 175 mg/week) and exhibited consistently high (>5.7 mg/L) serum warfarin concentrations. Altogether, these studies led to the hypothesis that VKORC1 is the site of action of warfarin and indicate that VKORC1 sequence is an important determinant of the warfarin dose response. However, such rare mutations do not account for the majority of warfarin-treated subjects and thus cannot explain the significantly high inter-individual and inter-population variability in warfarin dose requirements.

To characterize the genetic factors underlying warfarin sensitivity/resistance several research groups have conducted case-control association studies and found association between coumarin resistance or sensitivity and specific alleles of single nucleotide polymorphisms (SNPs) of the VKORC1 locus (the following VKORC1 SNPs designation correspond to GenBank Accession No. AY587020; SEQ ID NO:25). Thus, the C or T alleles of the 6484C/T polymorphism (rs9934438) were found to be associated with warfarin resistance or sensitivity, respectively (Rieder M J., et al., 2005; D'Andrea G, et al., 2005; Reitsma P H, et al., 2005; Geisen C, et al., 2005; Mushiroda T., et al., 2006); the G or A alleles of the 3673G/A polymorphism (rs17878363) were found to be associated with warfarin resistance or sensitivity, respectively (Rieder M J., et al., 2005; Bodin L., et al., 2005; Sconce et al., 2005; Yuan H-Y, et al., 2005); the C or G alleles of the 6853C/G polymorphism (rs8050894) were found to be associated with warfarin sensitivity or resistance, respectively (Veenstra D L., et al., 2005; Rieder M J., et al., 2005); and the 861C allele of the 861C/A polymorphism was associated with warfarin sensitivity (Veenstra D L., et al., 2005). However, Schelleman H., et al. (2007) examined the contribution of SNPs 1173C/T (6484C/T in SEQ ID NO:25) and −1639G/A (3673G/A in SEQ ID NO:25) in the VKORC1 gene to the variability in warfarin response, particularly in African Americans and concluded that these polymorphisms cannot be used for predicting over-anticoagulation among African Americans. In addition, Geisen C, et al. (2005b), identified a VKORC1 Asp36Tyr polymorphism in two patients with moderately increased coumarin requirement (i.e., 40-50 mg phenprocoumon per week) which were needed to maintain an INR between 2.0 and 3.0. However, to date, this polymorphism was not shown or suggested to be associated with coumarin resistance. U.S. Pat. Application Publication No. 2006/0084070 to Rieder et al., discloses the use of VKORC1 haplotypes H1-H9 for determining responsiveness for warfarin therapy.

SNPs in the VKORC1 gene were found to be ethnically stratified. For example, Takahashi H., et al., 2006, found that the VKORC1 6484T allele is more frequent among Japanese (89.1%) than among either Caucasians (42.2%) or African Americans (8.6%). In addition, the AA genotype of SNP 3673G/A was found to be the common genotype in the general Chinese population but not in the general Caucasian population (Yuan H-Y, et al., 2005). Moreover, the VKORC1*1 haplotype, which is considered a putative ancestral haplotype was found to be a common haplotype in individuals of an African descent (e.g., of the African or African American populations) (Geisen C et al 2005a).

The established notion however suggests that in the Caucasians, Afro-American and Chinese populations polymorphisms in CYP2C9 and VKORC1 genes together with the age factor can explain more than 50% of the variation leading to lower warfarin dose requirement (Bodin L, et al., 2005; Sconce E A, et al., 2005). In addition, it has been suggested that polymorphisms in γ-glutamyl carboxylase (GGCX), another component of vitamin K cycle, may have minor effects on warfarin dose response (Shikata E, et al., 2004; Wadelius M, et al., 2005; Loebstein R, et al., 2005). Recently, Wadelius M et al. (2007) concluded that polymorphisms in VKORC1, CYP2C9, Protein C (PROC) as well as non-genetic factors such as age, weight, drug interactions and indication for treatment account for up to 62% of warfarin dose requirement.

The present inventors have previously demonstrated that the microsomal epoxide hydrolase (mEH or EPHX1), a putative co-factor of VKOR, is another modulator of individual warfarin dose requirement (Loebstein R, et al., 2005). Specifically, the EPHX1 612T→C polymorphism (according to GenBank Accession No. NM_{—}000120.2; rs1051740) was found to be associated with the requirement of warfarin doses greater than 50 mg/week (>7 mg/day) independent of the effect of CYP2C9. This finding was further confirmed by Wadelius M et al 2007.

In addition, the endoplasmic reticulum Ca²⁺ binding protein calumenin (CALU), a putative inhibitor of VKOR and GGCX, was studied in rats for its possible involvement in warfarin sensitivity (Wallin R, et al., 2001; Wajih N, et al., 2004). Moreover, recent studies performed by the present inventors have shown that the R4Q polymorphism in the CALU gene is associated with higher warfarin doses, particularly a double dosage (i.e., homozygosity) of 4Q protein polymorph may underlie warfarin resistance (Vecsler M, et al., 2006).

Altogether, the prior art data suggest that polymorphisms in CYP2C9 and VKORC1 genes along with the age and weight factors may explain only 62% of the variations in coumarin dose requirement (see also FIG. 3).

There is thus a widely recognized need for, and it would be highly advantageous to have, a comprehensive diagnostic platform that enables high predictability of coumarin resistance and dose requirements.

### SUMMARY OF THE INVENTION

According to one aspect of the present invention there is provided a method of determining if an individual is predisposed to coumarin resistance, the method comprising determining in a sample of the individual a presence or an absence, in a homozygous or a heterozygous form of a thymidine nucleotide-containing allele at position 5417 of a VKORC1 polynucleotide as set forth in SEQ ID NO:25 and/or a tyrosine residue-containing polymorph at position 36 of a VKORC1 polypeptide as set forth in SEQ ID NO:28, thereby determining if the individual is predisposed to coumarin resistance.

According to another aspect of the present invention there is provided a kit for determining if an individual is predisposed to coumarin resistance, the kit comprising at least one reagent for determining a presence or an absence in a homozygous or a heterozygous form of a thymidine nucleotide-containing allele at position 5417 of a VKORC1 polynucleotide as set forth in SEQ ID NO:25 and/or a tyrosine residue-containing polymorph at position 36 of a VKORC1 polypeptide as set forth in SEQ ID NO:28.

According to yet another aspect of the present invention there is provided a method of predicting a responsiveness of an individual to coumarin treatment, comprising detecting in a sample of the individual a presence or an absence, in a homozygous or a heterozygous form of a thymidine nucleotide-containing allele at position 5417 of a VKORC1 polynucleotide as set forth in SEQ ID NO:25 and/or a tyrosine residue-containing polymorph at position 36 of a VKORC1 polypeptide as set forth in SEQ ID NO:28, thereby predicting the responsiveness of the individual to coumarin treatment.

According to still another aspect of the present invention there is provided a kit for predicting a responsiveness of an individual to coumarin treatment, the kit comprising at least one reagent for determining a presence or an absence in a homozygous or a heterozygous form of a thymidine nucleotide-containing allele at position 5417 of a VKORC1 polynucleotide as set forth in SEQ ID NO:25 and/or a tyrosine residue-containing polymorph at position 36 of a VKORC1 polypeptide as set forth in SEQ ID NO:28.

According to an additional aspect of the present invention there is provided a method of determining if an individual is suitable for genotype analysis of VKORC1 D36Y-related coumarin resistance, comprising determining in a sample of the individual a presence or an absence, in a homozygous or a heterozygous form of a VKORC1*1 haplotype, wherein said presence of said VKORC1*1 haplotype is indicative of the individual being suitable for genotype analysis of the VKORC1 D36Y-related coumarin resistance.

According to yet an additional aspect of the present invention there is provided a kit for determining if an individual is suitable for genotype analysis of VKORC1 D36Y-related coumarin resistance, the kit comprising at least one reagent for determining a presence or an absence in a homozygous or a heterozygous form of a VKORC1*1 haplotype.

According to further features in preferred embodiments of the invention described below, the coumarin is warfarin.

According to still further features in the described preferred embodiments the individual is predisposed to thromboembolism.

According to still further features in the described preferred embodiments the kit further comprising packaging material packaging at least one reagent and a notification in or on the packaging material, the notification identifying the kit for use in determining if the individual is predisposed to coumarin resistance.

According to still further features in the described preferred embodiments the kit further comprising packaging material packaging at least one reagent and a notification in or on the packaging material, the notification identifying the kit for use in predicting a responsiveness of an individual to coumarin treatment.

According to still further features in the described preferred embodiments the kit further comprising packaging material packaging at least one reagent and a notification in or on said packaging material, said notification identifying the kit for use in determining if an individual is suitable for genotype analysis of VKORC1 D36Y-related coumarin resistance.

According to still further features in the described preferred embodiments a presence of the thymidine nucleotide-containing allele at position 5417 of the VKORC1 polynucleotide and/or the tyrosine residue-containing polymorph at position 36 of the VKORC1 polypeptide is indicative of increased predisposition to coumarin resistance.

According to still further features in the described preferred embodiments the at least one reagent is at least one oligonucleotide capable of specifically hybridizing with a thymidine nucleotide-containing allele or a guanine nucleotide-containing allele at position 5417 of the VKORC1 polynucleotide.

According to still further features in the described preferred embodiments determining the presence or absence of the thymidine nucleotide-containing allele at position 5417 of the VKORC1 polynucleotide is effected by a method selected from the group consisting of: DNA sequencing, restriction fragment length polymorphism (RFLP analysis), allele specific oligonucleotide (ASO) analysis, Denaturing/Temperature Gradient Gel Electrophoresis (DGGE/TGGE), Single-Strand Conformation Polymorphism (SSCP) analysis, Dideoxy fingerprinting (ddF), pyrosequencing analysis, acycloprime analysis, Reverse dot blot, GeneChip microarrays, Dynamic allele-specific hybridization (DASH), Peptide nucleic acid (PNA) and locked nucleic acids (LNA) probes, TaqMan, Molecular Beacons, Intercalating dye, FRET primers, AlphaScreen, SNPstream, genetic bit analysis (GBA), Multiplex minisequencing, SNaPshot, MassEXTEND, MassArray, GOOD assay, Microarray miniseq, arrayed primer extension (APEX), Microarray primer extension, Tag arrays, Coded microspheres, Template-directed incorporation (TDI), fluorescence polarization, Colorimetric oligonucleotide ligation assay (OLA), Sequence-coded OLA, Microarray ligation, Ligase chain reaction, Padlock probes, Rolling circle amplification, Sequenom Mass Spectrograph and Invader assay.

According to still further features in the described preferred embodiments the at least one reagent is an antibody capable of differentially binding at least one polymorph of the Aspartic acid residue-containing polymorph or the tyrosine residue-containing polymorph at position 36 of the VKORC1 polypeptide.

According to still further features in the described preferred embodiments the at least one reagent is an antibody capable of differentially binding the tyrosine residue-containing polymorph at position 36 of the VKORC1 polypeptide.

According to still further features in the described preferred embodiments determining the presence or absence of the tyrosine residue-containing polymorph is effected by an antibody capable of differentially binding at least one polymorph of an Aspartic acid residue-containing polymorph or a the tyrosine residue-containing polymorph at position 36 of the VKORC1 polypeptide.

According to still further features in the described preferred embodiments determining the presence or absence of the tyrosine residue-containing polymorph is effected by an antibody capable of differentially binding the tyrosine residue-containing polymorph at position 36 of the VKORC1 polypeptide.

According to still further features in the described preferred embodiments the sample of the individual is a DNA sample.

According to still further features in the described preferred embodiments the sample of the individual is a protein sample.

According to still further features in the described preferred embodiments the individual carries the VKORC1*1 haplotype.

According to still further features in the described preferred embodiments the individual is of a population selected from the group consisting of an African population, an African American population, a Jewish Ethiopian population, an Ashkenazi Jewish population, Caucassian population and an Indian population.

According to still further features in the described preferred embodiments the VKORC1*1 haplotype comprises the guanine nucleotide-containing allele at position 514 of SEQ ID NO:37, the guanine nucleotide-containing allele at position 941 of SEQ ID NO:25 and the guanine nucleotide-containing allele at position 256 of SEQ ID NO:38.

According to still further features in the described preferred embodiments the at least one reagent is at least one oligonucleotide capable of specifically hybridizing with a guanine nucleotide-containing allele at position 514 of SEQ ID NO:37, a guanine nucleotide-containing allele at position 941 of SEQ ID NO:25 and/or a guanine nucleotide-containing allele at position 256 of SEQ ID NO:38.

The present invention successfully addresses the shortcomings of the presently known configurations by providing a methods and kits for determining if an individual is predisposed to coumarin resistance, for predicting a responsiveness of an individual to coumarin treatment and for determining if an individual is suitable for genotype analysis of VKORC1 D36Y-related coumarin resistance.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a schematic illustration depicting the components of the vitamin K redox cycle. Reduced vitamin K (Vit. KH₂) is a cofactor required for the activation of clotting factors II, VII, IX and X, and proteins C, S and Z by γ-glutamyl carboxylase (GGCX) resulting in oxidized vitamin K (Vit. KO). Regeneration of Vit. KH₂ is catalyzed by the vitamin K 2,3-epoxide reductase (VKOR), a complex enzyme consisting of VKORC1 subunit and other cofactors such as microsomal epoxide hydrolase (EPHX1) and a putative cofactor glutathione S-transferase A1 (GSTA1). The endoplasmic reticulum Ca²⁺ binding protein calumenin (CALU) has been suggested to modulate the activity of VKOR and GGCX. Warfarin interferes with the vitamin K cycle by inhibiting VKOR and limiting the regeneration of Vit. KH₂, and thus limiting the production of active clotting factors. The S-warfarin is metabolized by the cytochrome P450 CYP2C9;

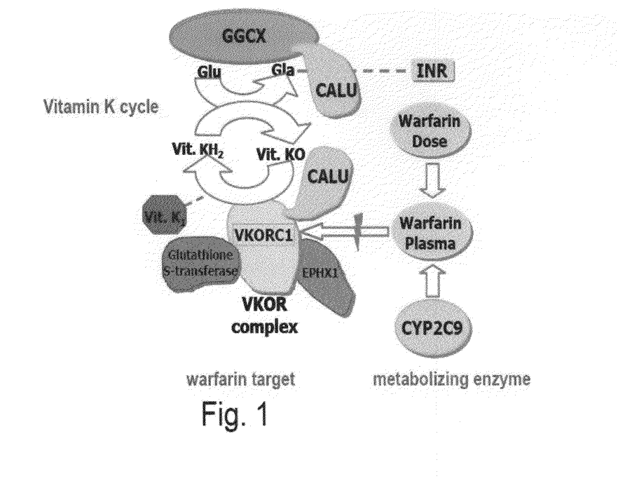

FIG. 2 is a bar graph depicting the effect of combined genotypes of the CYP2C9, VKORC1 and CALU genes on warfarin dose requirements. Bars represent daily dose (means±standard error) for each combined genotype subgroups. The combined genotypes are ordered according to the model proposed by Wallin R, et al., 2001, assuming the reduced VKORC1 activity associated with the VKORC1 6853G→C polymorphism and the reduced activity of CALU associated with the CALU 73G→A (R4Q) polymorphism and assigning equal weight to CYP2C9 and VKORC1 genotypes. The dotted line represents the median weight-normalized warfarin dose of 4.8 mg/day. *p<0.05; ** p<0.01 by Scheffe (ANOVA F=5.4, p<0.0001); wt=wild-type homozygotes; mu=mutant heterozygotes+homozygotes; n=number of subjects in subgroup; Wt/wt/mu=CYP2C9*1/VKORC1-6853G/CALU-73A; mu/wt/mu=CYP2C9*2 and *3/NKORC1-6853G/CALU-73A; wt/wt/wt=CYP2C9*1/VKORC1-6853G/CALU-73G; mu/wt/wt=CYP2C9*2 and *3/VKORC1-6853 G/CALU-73G; wt/mu/wt=CYP2C9*1 VKORC1-6853C/CALU-73G; wt/mu/mu=CYP2C9*1/VKORC1-6853C/CALU-73A; mu/mu/mu ═CYP2C9*2 and *3/NKORC1-6853C/CALU-73A; mu/mu/wt=CYP2C9*2 and *3NKORC1-6853C/CALU-73G. CYP2C9=cytochrome P450 2C9; VKORC1=vitamin K epoxide reductase component 1; CALU=calumenin;

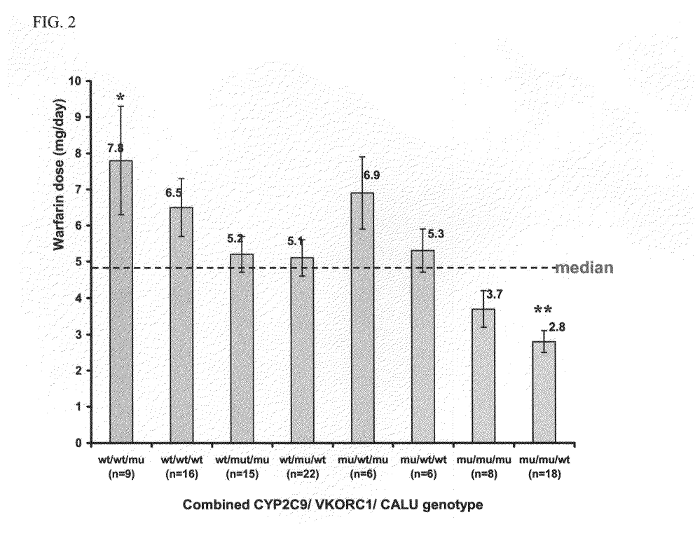

FIG. 3 is a pie-like presentation depicting the current knowledge of the relative contribution of predictors of individual warfarin dose requirements. Genetic factors including the known CYP2C9 variants (include CYP2C9*1/*2/*3) and VKORC1 intron 1 variant (Tag-SNP of VKORC1*2) polymorphisms associated with warfarin sensitivity explain about 41% of inter-individual variability in warfarin dose. Age, weight and dietary vitamin K intake explain additional 21.7% of the variability. The remaining 37.3% may include additional genetic factors underlying pharmacokinetics and pharmacodynamic of warfarin mechanism;

FIG. 4 is a pie-like presentation depicting the relative contribution of predictors of individual warfarin dose requirements following the teachings of the present invention (i.e., including the VKORC1 D36Y polymorphism). Factors contributing to individual warfarin dose requirements include age, weight and genetic variants of the CYP2C9 and VKORC1 genes including CYP2C9*1/*2/*3 variants and the VKORC1*1/*2/*3 and the VKORC1 exon 1 polymorphism (5417G→T according to SEQ ID NO 25; D36Y) which is associated with higher warfarin doses. Note that the D36Y VKORC1 polymorphism accounts for 18% of variability of warfarin dose-response, which is more that the effects of CYP2C9 or known VKORC1 haplotypes, demonstrating that the D36Y VKORC1 polymorphism exhibits the highest contribution value and therefore is the most significant factor in warfarin dose requirement. The overall predictability of the presented factors is 62%.

### DESCRIPTION OF THE PREFERRED EMBODIMENTS

The present invention is of the high association of the 5417T allele of the 5417G/T SNP in the VKORC1 gene with high coumarin dose requirements and which can be used to determine the predisposition of an individual to coumarin resistance. Specifically, the present invention provides methods and kits for determining the predisposition of an individual to coumarin resistance and for predicting the responsiveness of an individual to coumarin treatment.

The principles and operation of the methods and kits of determining predisposition to coumarin resistance and predicting the responsiveness of an individual to coumarin treatment according to the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Coumarin derivatives such as warfarin, phenprocoumon and acenocoumarol (AC) are the most common anticoagulants prescribed worldwide for the prevention of thromboembolism. However, due to an extensive variability in individual response to coumarin derivatives (e.g., about 20-fold variability in the response to warfarin), their use requires careful clinical management to balance the risks of over-anticoagulation and bleeding with those of under-anticoagulation and blood clotting. Factors known to affect coumarin dose requirements include genetic factors such as the CYP2C9*2 and *3 variants that are associated with coumarin sensitivity, the VKORC1 6484T, 3673A, 6853C and/or 861C alleles which are associated with coumarin sensitivity, and the CALU 4Q polymorphic variant that is associated with coumarin resistance. Recently, Geisen et al. 2005b, identified a VKORC1 Asp36Tyr polymorphism in two patients with moderately increased coumarin requirement (i.e., 40-50 mg phenprocoumon per week) which were needed to maintain an iNR between 2.0 and 3.0. In addition, U.S. Pat. Application Publication No. 2006/0084070 to Rieder et al., discloses the use of VKORC1 haplotype H1-H9 for determining responsiveness for warfarin therapy.

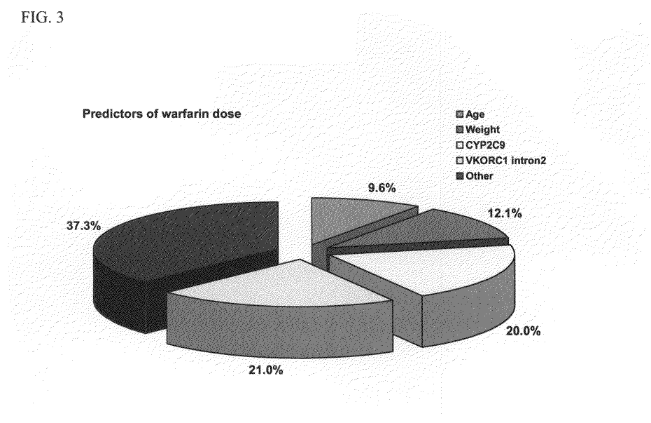

Other, non-genetic factors affecting coumarin dose requirements include age, weight, dietary vitamin K intake, certain diseases (e.g., heart failure or severe liver disease), medications (e.g., procor or aspirin) and ethnic origin (e.g., Chinese and Japanese require lower doses of warfarin as compared to Caucasians and African Americans). Altogether, the currently available data can explain up to 62% of the variations in coumarin dose requirements (see for example, FIG. 3).

While reducing the present invention to practice, the present inventors have uncovered that the 5417G→T [numbering corresponds to SEQ ID NO:25 (GenBank Accession No. AY587020)] non-synonymous SNP in the VKORC1 gene, which results in the D36Y substitution (numbering according to SEQ ID NO:28) is highly associated with coumarin resistance.

As is shown in Table 4 and is described in Example 1 of the Examples section which follows, while the VKORC1 5417T allele was highly frequent among a selected group of warfarin resistance subjects (7/30 chromosomes; 23%) who require average weekly doses of 112.8 mg warfarin (range 80-185 mg/week), this allele was absent in a selected group of warfarin sensitive subjects (0/16 chromosomes) who require average weekly doses of 10.5 mg (range 7-13 mg/week). Further analysis of a group of 99 unselected warfarin-treated subjects who require various doses of warfarin (mean 39.4±7.8 mg/week, range 8-105 mg/week) revealed that the overall frequency of the VKORC1 5417T allele in this group is 4% (8/198 chromosomes), all of them were in the upper quartile >75% of warfarin dose requirements (>70 mg/week; mean dose 80.9±10.1 mg/week) compared to the other 91 patients lacking this polymorphism (who require a mean dose of 42.7±7.5 mg/week, range 8-70 mg/week; see also Loebstein R., 2007). Moreover, the D36Y polymorphism was significantly over-represented in the Ethiopian Jewish population (15%) as compared to other Jewish or non-Jewish populations (Table 6, Example 2 of the Examples section which follows). In addition, as is shown in Table 9 and described in Example 3 of the Examples section which follows, the D36Y polymorphism was found to co-present with the tag-SNP of the VKORC1*1 haplotype, i.e., the haplotype comprising the wild-type alleles of the Tag-SNPs of VKORC1*2 (the G allele at position 514 of SEQ ID NO:37), VKORC1*3 (the G allele at position 941 of SEQ ID NO:25) and VKORC1*4 (the G allele on the + strand (or the C allele on the − strand) at position 256 of SEQ ID NO:38) which is a common haplotype (about 31%) in individuals of the African descent (Geisen C et al 2005).

Thus, according to one aspect of the present invention there is provided a method of determining if an individual is predisposed to coumarin resistance. The method is effected by determining in a sample of the individual a presence or an absence, in a homozygous or a heterozygous form of a thymidine nucleotide-containing allele at position 5417 of a VKORC1 polynucleotide as set forth in SEQ ID NO:25 and/or a tyrosine residue-containing polymorph at position 36 of a VKORC1 polypeptide as set forth in SEQ ID NO:28.

The term “individual” as used herein, refers to a mammal, preferably a human being. Preferably, this term encompasses individuals who are treated with or are suggested for treatment with anticoagulants such as coumarin. Non-limiting examples of such individuals include those who suffer from atrial fibrillation, deep venous thrombosis (DVT), pulmonary thromboembolism (PTE), hereditary thrombophilias, antiphospholipid syndrome (APLA), several dilated myopathies or individuals who underwent implantation of prosthetic heart valve.

The term “coumarin” as used herein refers to coumarin derivatives such as warfarin, phenprocoumon and acenocoumarol (AC).

The phrase “coumarin resistance” as used herein, refers to a condition of an individual who requires relatively high doses of coumarin so as to achieve the desired antithrombotic effect of coumarin and is thus exposed to coumarin-related side effects [e.g., bleeding (in urine, stool, vomit, coughing, gums), sudden leg or foot pain and/or dizziness]. It will be appreciated that coumarin resistance is an opposite condition to coumarin sensitivity, in which an individual treated with coumarin requires relatively low doses of coumarin to achieve the antithrombotic effect of coumarin while being exposed to the risk of bleeding. As described in the background section hereinabove, coumarin dose requirements are highly variable between individuals of the same ethnic group as well as between different ethnic groups. Thus, the mean warfarin maintenance doses is 3.3 mg/day (i.e., 23 mg/week) in the Japanese and Chinese population, 5.0 mg/day (i.e., 35 mg/week) in the Caucasian population and higher than 5 mg/day (i.e., >35 mg/week) in the African American and Indian population (see for example, Takahashi et al., 2003; Zhao et al., 2004; Absher et al., Ann Pharmacother 2002; Dang et al., 2005; and Gan G G., 2003).

According to one preferred embodiment of this aspect of the present invention, coumarin resistance refers to subjects requiring coumarin (e.g., warfarin) doses that are higher than 56 mg coumarin/week, more preferably, higher than 63 mg coumarin/week, more preferably, higher than 70 mg coumarin/week, more preferably, higher than 77 mg coumarin/week, more preferably, higher than 84 mg coumarin/week, more preferably, higher than 105 mg coumarin/week, more preferably, between 70-300 mg coumarin/week, more preferably, between 70-200 mg coumarin/week, e.g., about 180 mg coumarin/week.

It will be appreciated that the doses of coumarin derivatives required by the individual are adjusted prior to and during treatment by monitoring the presence and/or level of various markers such as blood coagulation factors and vitamin K dependent clotting factors, protein in the absence of vitamin K (PVKA). Generally, the coumarin dose requirements are adjusted using the standard International Normalized Ratio (INR) values; optimal INR values are between 2-3 for atrial fibrillation, deep vein thrombosis and pulmonary embolism, and between 2.5-3.5 for artificial heart valves and APLA syndrome.

Preferably, determining the predisposition to coumarin resistance is effected prior to or during the vulnerable period of coumarin intake. As used herein the “vulnerable period of coumarin intake” refers to the period during which the subject who requires coumarin as an antithrombotic agent has an increased risk of re-thromobosis in case an under-dose of coumarin is provided thereto, or an increased risk for bleeding and related conditions (e.g., hemorrhages, hematoma) in case an over-dose of coumarin is provided thereto. The vulnerable period of coumarin intake begins with the initiation of the treatment immediately after the first dose of coumarin is provided to the individual and lasts until the individual reaches the stable state therapeutic state INR=2.0-3.0, which is an indeterminate period between 2 weeks and 2 months.

As used herein, the term “predisposed” when used with respect to coumarin resistance refers to an individual which is more likely to develop coumarin resistance upon coumarin treatment (i.e., to require high doses of coumarin to achieve the antithrombotic effect) than a non-predisposed individual.

As used herein the phrase “VKORC1 polynucleotide” refers to the DNA sequence on chromosome 16p11.2 of the human genome encoding subunit 1 of the vitamin K epoxide reductase (VKOR) enzyme [genomic sequence—GenBank Accession No. NC_{—}000016 positions 31009676-31013777; coding sequence—GenBank Accession No. AY587020 (SEQ ID NO:25)]. The VKORC1 enzyme is responsible for reducing vitamin K 2,3-epoxide to the enzymatically activated form (i.e., the reduced form) which is required for the carboxylation of glutamic acid residues in some blood-clotting proteins (e.g., factors II, VII, IX and X). The VKORC1 gene is subject to alternative splicing resulting in two alternatively spliced transcripts (GenBank Accession Nos. NM_{—}024006.4 and NM_{—}206824.1) which encode different VKORC1 isoforms, i.e., isoform 1 (as set forth by GenBank Accession No. AAS83106) and isoform 2 (as set forth GenBank Accession No. NP_{—}996560)].

As used herein the phrase “VKORC1 polypeptide” refers to the polypeptide of isoform 1 of VKORC1 as set forth by GenBank Accession No. AAS83106 (SEQ ID NO:28).

As described in the Background section hereinabove, genetic polymorphisms in the VKORC1 gene such as 6853C/G, 9041G/A, 3673G/A, 6484C/T, 861C/A, 5808T/G or 5432G/T (numbering of polymorphic nucleotides correspond to SEQ ID NO:25) were found to be associated with variability in coumarin dose requirement.

The term “polymorphism” refers to the occurrence of two or more genetically determined variant forms (alleles) of a particular nucleic acid (or nucleic acids) of a nucleic acid sequence (e.g., gene) at a frequency where the rarer (or rarest) form could not be maintained by recurrent mutation alone. Polymorphisms can arise from deletions, insertions, duplications, inversions, substitution and the like of one or more nucleic acids. Preferably, the polymorphism used by the present invention is a single nucleotide polymorphism (SNP) which comprises the G/T substitution at position 5417 of the VKORC1 gene (SEQ ID NO:25, GenBank Accession No. AY587020). Such SNP is a non-synonymous polymorphism (i.e., results in an amino acid change in the translated protein) which comprises the D36Y substitution (i.e., a substitution of an aspartic acid residue with a tyrosine residue at position 36) of the VKORC1 polypeptide set forth by SEQ ID NO:28.

The terms “homozygous” or “heterozygous” refer to two identical or two different alleles and/or protein polymorphs, respectively, of a certain polymorphism.

The term “absence” as used herein with respect to the allele and/or the protein polymorph describes the negative result of a specific polymorphism determination test. For example, if the polymorphism determination test is suitable for the identification of a thymidine nucleotide-containing allele at position 5417 of the VKORC1 polynucleotide as set forth in SEQ ID NO:25, and the individual on which the test is performed is homozygote for the guanine nucleotide-containing allele at position 5417 of the VKORC1 polynucleotide, then the result of the test will be “absence of the thymidine nucleotide-containing allele”. Similarly, if the polymorphism determination test is suitable for the identification of a tyrosine residue-containing polymorph at position 36 of the VKORC1 polypeptide as set forth in SEQ ID NO:28, and the individual on which the test is performed is homozygote for the aspartic acid-containing polymorph at position 36 of the VKORC1 polypeptide, then the result of the test will be “absence of the tyrosine residue-containing polymorph”.

The predisposition to coumarin resistance can be quantified by generating and using genotype relative risk (GRR) values. The GRR is the increased chance of an individual with a particular genotype (or protein polymorph) to be resistant to coumarin (i.e., to require high doses of coumarin) when used as anticoagulant. Thus, the GRR of the risk genotype G, with respect to the protective genotype G₀, is the ratio between the risk of an individual carrying genotype G to become coumarin resistant, and the risk of an individual carrying genotype G₀ to become coumarin resistant. The GRR used herein is represented in terms of an appropriate odds ratio (OR) of G versus G₀ in cases and controls. Moreover, computation of GRR of haplotypes is based on a multiplicative model in which the GRR of an homozygote individual is the square of the GRR of an heterozygote individual. For further details see Risch and Merikangas, 1996 [The future of genetic studies of complex human diseases. Science 273: 1516-1517].

The odds ratio (OR) is an estimate of the relative risk, i.e., the increased probability of being coumarin resistance in populations exposed to the risk allele. OR and approximate confidence intervals (C.I.) can be computed in a standard way [Alan Agresti (1990). Categorical data analysis. New York: Wiley, pp. 54-55] in order to examine the structure and strength of association between the genotype and coumarin resistance.

Once calculated, the GRR can reflect the predisposition risk of an individual with a specific VKORC1 genotype (i.e., with the G or T alleles and/or the GG, GT or TT genotypes of the VKORC15417G/T polymorphism) to develop coumarin resistance upon treatment with coumarin derivatives.

For example, as is shown in Example 2 of the Examples section which follows, in the unselected group of 99 warfarin-treated subjects the calculated OR (i.e., the increased relative risk) of the 5417T allele (i.e., the risk allele) was found to be 13.0 [95% C.I. 1.3-124.2].

The GRR can be further used to calculate the population attributable risk (PAR), i.e., the percentage of cases that would not have been affected if the population was monomorphic for the protective allele and genotype. The PAR value of a certain allele is calculated by the following equation: (K−1)/K, wherein K is Σfᵢ·gᵢ, fᵢ is the frequency of the i genotype or double genotype and gᵢ is the estimated GRR of the i genotype or double genotype, respectively.

As mentioned hereinabove, the thymidine nucleotide-containing allele at position 5417 of VKORC1 as set forth in SEQ ID NO:25 is linked to the VKORC1*1 haplotype.

As used herein the phrase “VKORC1*1 haplotype” refers to the wild-type haplotype of the VKORC1 gene which comprises at least a guanine nucleotide-containing allele at position 514 of SEQ ID NO:37, a guanine nucleotide-containing allele at position 941 of SEQ ID NO:25 and a guanine nucleotide-containing allele at position 256 of SEQ ID NO:38.

Preferably, determining the presence or absence of the thymidine nucleotide-containing allele at position 5417 of a VKORC1 polynucleotide as set forth in SEQ ID NO:25 is performed in sample of an individual who carries the VKORC1*1 haplotype. Non-limiting examples of individuals who carry the VKORC1*1 haplotype include individuals of the African population, African American population, Jewish Ethiopian population and Ashkenazi Jewish population.

It will be appreciated that the 5417T allele of VKORC1 as set forth in SEQ ID NO:25 can be also detected in individuals of the Caucassian population.

In addition, and without being bound by any theory, since individuals of the Indian population are also known to require high doses of coumarin (Gan G G., et al., 2003; and Zhao F et al 2004) such individuals are likely to carry the VKORC1*1 haplotype and/or the 5417T allele of VKORC1 as set forth in SEQ ID NO:25.

The present invention further envisages a method of screening for subjects who are at risk of having coumarin resistance due to the 5417T allele of VKORC1.

Thus, according to another aspect of the present invention there is provided a method of determining if an individual is suitable for genotype analysis of VKORC1 D36Y-related coumarin resistance. The method is effected by determining in a sample of the individual a presence or an absence, in a homozygous or a heterozygous form of a VKORC1*1 haplotype, wherein said presence of said VKORC1*1 haplotype is indicative of the individual being suitable for genotype analysis of the VKORC1 D36Y-related coumarin resistance.

As used herein the phrase “VKORC1 D36Y-related coumarin resistance” refers to coumarin resistance which is associated with the 5417T allele of VKORC1 as set forth by SEQ ID NO:25 or the tyrosine residue-containing polymorph at position 36 of a VKORC1 polypeptide as set forth in SEQ ID NO:28.

Determining the presence or the absence of the VKORC1 5417T allele, the VKORC1 Y36 polymorph and/or the VKORC1*1 haplotype according to this aspect of the present invention can be effected using a DNA and/or a protein sample which is derived from any suitable biological sample of the individual, including, but not limited to, blood, plasma, blood cells, saliva or cells derived by mouth wash, and body secretions such as urine and tears, and from biopsies, etc. Additionally or alternatively, nucleic acid tests can be performed on dry samples (e.g. hair or skin). Methods of extracting DNA and protein samples from blood samples are well known in the art.

The VKORC1 5417G/T SNP of the VKORC1 polynucleotide and/or the VKORC1*1 haplotype can be identified using a variety of approaches suitable for identifying sequence alterations. One option is to determine the entire gene sequence of a PCR reaction product (e.g., using the PCR primers set forth by SEQ ID NOs:1 and 2 and the PCR conditions described in Example 1 of the Examples section which follows). Alternatively, a given segment of nucleic acids may be characterized on several other levels. Following is a non-limiting list of SNP detection methods which can be used to identify the VKORC1 5417G/T SNP and/or the VKORC1*1 haplotype of the present invention.

Restriction fragment length polymorphism (RFLP): This method uses a change in a single nucleotide (the SNP nucleotide) which modifies a recognition site for a restriction enzyme resulting in the creation or the destruction of an RFLP.

For example, RFLP can be used to detect the VKORC1 5417T allele in a genomic DNA of an individual. Briefly, genomic DNA is amplified using the VKORC1 forward 5′-CTCCGTGGCTGGTTTTCT (SEQ ID NO:1) and reverse 5′-CCGATCCCAGACTCCAGAAT (SEQ ID NO:2) PCR primers and the resultant 303 bp PCR product is further subjected to digestion using a restriction enzyme such as RsaI which is capable of differentially digesting a PCR product containing the T allele (and not the G allele) at position 5417 of SEQ ID 25, resulting in two fragments of 155 and 148 bp (see application of RFLP analysis in Example 2 of the Examples section which follows).

Single nucleotide mismatches in DNA heteroduplexes are also recognized and cleaved by some chemicals, providing an alternative strategy to detect single base substitutions, generically named the “Mismatch Chemical Cleavage” (MCC) (Gogos et al., Nucl. Acids Res., 18:6807-6817, 1990). However, this method requires the use of osmium tetroxide and piperidine, two highly noxious chemicals which are not suited for use in a clinical laboratory.

Allele specific oligonucleotide (ASO): In one embodiment, this method uses an allele-specific oligonucleotide (ASO) which is designed to hybridize in proximity to the polymorphic nucleotide, such that a primer extension or ligation event can be used as the indicator of a match or a mis-match. In another embodiment, the ASO is used as a hybridization probe, which due to the differences in the melting temperature of short DNA fragments differing by a single nucleotide, is capable of differentially hybridizing to a certain allele of the SNP and not to the other allele. It will be appreciated that stringent hybridization and washing conditions are preferably employed. Hybridization with radioactively labeled ASO also has been applied to the detection of specific SNPs (Conner et al., Proc. Natl. Acad. Sci., 80:278-282, 1983).

Suitable ASO probes which can be used along with the present invention to identify the presence of the VKORC15417G/T polymorphism include the 5′-GGTACCGGGATTACCG probe (SEQ ID NO:36; Tm=56.6° C.) which can differentially hybridize to the VKORC1 5417T allele and the 5′-GGGACCGGGATTACCG probe (SEQ ID NO:31; Tm=61.8° C.) which can differentially hybridize to the VKORC1 5417G allele.

Denaturing/Temperature Gradient Gel Electrophoresis (DGGE/TGGE): Two other methods rely on detecting changes in electrophoretic mobility in response to minor sequence changes. One of these methods, termed “Denaturing Gradient Gel Electrophoresis” (DGGE) is based on the observation that slightly different sequences will display different patterns of local melting when electrophoretically resolved on a gradient gel. In this manner, variants can be distinguished, as differences in melting properties of homoduplexes versus heteroduplexes differing in a single nucleotide can detect the presence of SNPs in the target sequences because of the corresponding changes in their electrophoretic mobilities. The fragments to be analyzed, usually PCR products, are “clamped” at one end by a long stretch of G-C base pairs (30-80) to allow complete denaturation of the sequence of interest without complete dissociation of the strands. The attachment of a GC “clamp” to the DNA fragments increases the fraction of mutations that can be recognized by DGGE (Abrams et al., Genomics 7:463-475, 1990). Attaching a GC clamp to one primer is critical to ensure that the amplified sequence has a low dissociation temperature (Sheffield et al., Proc. Natl. Acad. Sci., 86:232-236, 1989; and Lerman and Silverstein, Meth. Enzymol., 155:482-501, 1987). Modifications of the technique have been developed, using temperature gradients (Wartell et al., Nucl. Acids Res., 18:2699-2701, 1990), and the method can be also applied to RNA:RNA duplexes (Smith et al., Genomics 3:217-223, 1988).

Limitations on the utility of DGGE include the requirement that the denaturing conditions must be optimized for each type of DNA to be tested. Furthermore, the method requires specialized equipment to prepare the gels and maintain the needed high temperatures during electrophoresis. The expense associated with the synthesis of the clamping tail on one oligonucleotide for each sequence to be tested is also a major consideration. In addition, long running times are required for DGGE. The long running time of DGGE was shortened in a modification of DGGE called constant denaturant gel electrophoresis (CDGE) (Borrensen et al., Proc. Natl. Acad. Sci. USA 88:8405, 1991). CDGE requires that gels be performed under different denaturant conditions in order to reach high efficiency for the detection of SNPs.

A technique analogous to DGGE, termed temperature gradient gel electrophoresis (TGGE), uses a thermal gradient rather than a chemical denaturant gradient (Scholz, et al., Hum. Mol. Genet. 2:2155, 1993). TGGE requires the use of specialized equipment which can generate a temperature gradient perpendicularly oriented relative to the electrical field. TGGE can detect mutations in relatively small fragments of DNA therefore scanning of large gene segments requires the use of multiple PCR products prior to running the gel.

Single-Strand Conformation Polymorphism (SSCP): Another common method, called “Single-Strand Conformation Polymorphism” (SSCP) was developed by Hayashi, Sekya and colleagues (reviewed by Hayashi, PCR Meth. Appl., 1:34-38, 1991) and is based on the observation that single strands of nucleic acid can take on characteristic conformations in non-denaturing conditions, and these conformations influence electrophoretic mobility. The complementary strands assume sufficiently different structures that one strand may be resolved from the other. Changes in sequences within the fragment will also change the conformation, consequently altering the mobility and allowing this to be used as an assay for sequence variations (Orita, et al., Genomics 5:874-879, 1989).

The SSCP process involves denaturing a DNA segment (e.g., a PCR product) that is labeled on both strands, followed by slow electrophoretic separation on a non-denaturing polyacrylamide gel, so that intra-molecular interactions can form and not be disturbed during the run. This technique is extremely sensitive to variations in gel composition and temperature. A serious limitation of this method is the relative difficulty encountered in comparing data generated in different laboratories, under apparently similar conditions.

Dideoxy fingerprinting (ddF): The dideoxy fingerprinting (ddF) is another technique developed to scan genes for the presence of mutations (Liu and Sommer, PCR Methods Appli., 4:97, 1994). The ddF technique combines components of Sanger dideoxy sequencing with SSCP. A dideoxy sequencing reaction is performed using one dideoxy terminator and then the reaction products are electrophoresed on nondenaturing polyacrylamide gels to detect alterations in mobility of the termination segments as in SSCP analysis. While ddF is an improvement over SSCP in terms of increased sensitivity, ddF requires the use of expensive dideoxynucleotides and this technique is still limited to the analysis of fragments of the size suitable for SSCP (i.e., fragments of 200-300 bases for optimal detection of mutations).

Pyrosequencing™ analysis (Pyrosequencing, Inc. Westborough, Mass., USA): This technique is based on the hybridization of a sequencing primer to a single stranded, PCR-amplified, DNA template in the presence of DNA polymerase, ATP sulfurylase, luciferase and apyrase enzymes and the adenosine 5′ phosphosulfate (APS) and luciferin substrates. In the second step the first of four deoxynucleotide triphosphates (dNTP) is added to the reaction and the DNA polymerase catalyzes the incorporation of the deoxynucleotide triphosphate into the DNA strand, if it is complementary to the base in the template strand. Each incorporation event is accompanied by release of pyrophosphate (PPi) in a quantity equimolar to the amount of incorporated nucleotide. In the last step the ATP sulfurylase quantitatively converts PPi to ATP in the presence of adenosine 5′ phosphosulfate. This ATP drives the luciferase-mediated conversion of luciferin to oxyluciferin that generates visible light in amounts that are proportional to the amount of ATP. The light produced in the luciferase-catalyzed reaction is detected by a charge coupled device (CCD) camera and seen as a peak in a Pyrogram™. Each light signal is proportional to the number of nucleotides incorporated.

Acycloprime™ analysis (Perkin Elmer, Boston, Mass., USA): This technique is based on fluorescent polarization (FP) detection. Following PCR amplification of the sequence containing the SNP of interest, excess primer and dNTPs are removed through incubation with shrimp alkaline phosphatase (SAP) and exonuclease I. Once the enzymes are heat inactivated, the Acycloprime-FP process uses a thermostable polymerase to add one of two fluorescent terminators to a primer that ends immediately upstream of the SNP site. The terminator(s) added are identified by their increased FP and represent the allele(s) present in the original DNA sample. The Acycloprime process uses AcycloPol™, a novel mutant thermostable polymerase from the Archeon family, and a pair of AcycloTerminators™ labeled with R110 and TAMRA, representing the possible alleles for the SNP of interest. AcycloTerminator™ non-nucleotide analogs are biologically active with a variety of DNA polymerases. Similarly to 2′,3′-dideoxynucleotide-5′-triphosphates, the acyclic analogs function as chain terminators. The analog is incorporated by the DNA polymerase in a base-specific manner onto the 3′-end of the DNA chain, and since there is no 3′-hydroxyl, is unable to function in further chain elongation. It has been found that AcycloPol has a higher affinity and specificity for derivatized AcycloTerminators than various Taq mutant have for derivatized 2′,3′-dideoxynucleotide terminators.

Reverse dot blot: This technique uses labeled sequence specific oligonucleotide probes and unlabeled nucleic acid samples. Activated primary amine-conjugated oligonucleotides are covalently attached to carboxylated nylon membranes. After hybridization and washing, the labeled probe, or a labeled fragment of the probe, can be released using oligomer restriction, i.e., the digestion of the duplex hybrid with a restriction enzyme. Circular spots or lines are visualized colorimetrically after hybridization through the use of streptavidin horseradish peroxidase incubation followed by development using tetramethylbenzidine and hydrogen peroxide, or via chemiluminescence after incubation with avidin alkaline phosphatase conjugate and a luminous substrate susceptible to enzyme activation, such as CSPD, followed by exposure to x-ray film.

LightCycler™ Analysis (Roche, Indianapolis, Ind., USA)—The LightCycler™ instrument consists of a thermocycler and a fluorimeter component for on-line detection. PCR-products formed by amplification are detected on-line through fluorophores coupled to two sequence-specific oligonucleotide hybridization probes. One of the oligonucleotides has a fluorescein label at its 3′-end (donor oligonucleotide) and the other oligonucleotide is labeled with LightCyler™-Red 640 at its 5′-end (acceptor oligonucleotide). When both labeled DNA-probes are hybridized to their template, energy is transferred from the donor fluorophore to the acceptor fluorophore following the excitation of the donor fluorophore using an external light source with a specific wavelength. The light that is emitted by the acceptor fluorophore can be detected at a defined wavelength. The intensity of this light signal is proportional to the amount of PCR-product.

It will be appreciated that advances in the field of SNP detection have provided additional accurate, easy, and inexpensive large-scale SNP genotyping techniques, such as dynamic allele-specific hybridization (DASH, Howell, W. M. et al., 1999. Dynamic allele-specific hybridization (DASH). Nat. Biotechnol. 17: 87-8), microplate array diagonal gel electrophoresis [MADGE, Day, I. N. et al., 1995. High-throughput genotyping using horizontal polyacrylamide gels with wells arranged for microplate array diagonal gel electrophoresis (MADGE). Biotechniques. 19: 830-5], the TaqMan system (Holland, P. M. et al., 1991. Detection of specific polymerase chain reaction product by utilizing the 5′→3′ exonuclease activity of Thermus aquaticus DNA polymerase. Proc Natl Acad Sci USA. 88: 7276-80), as well as various DNA “chip” technologies such as the GeneChip microarrays (e.g., Affymetrix SNP chips) which are disclosed in U.S. Pat. No. 6,300,063 to Lipshutz, et al. 2001, which is fully incorporated herein by reference, Genetic Bit Analysis (GBA™) which is described by Goelet, P. et al. (PCT Appl. No. 92/15712), peptide nucleic acid (PNA, Ren B, et al., 2004. Nucleic Acids Res. 32: e42) and locked nucleic acids (LNA, Latorra D, et al., 2003. Hum. Mutat. 22: 79-85) probes, Molecular Beacons (Abravaya K, et al., 2003. Clin Chem Lab Med. 41: 468-74), intercalating dye [Germer, S, and Higuchi, R. Single-tube genotyping without oligonucleotide probes. Genome Res. 9:72-78 (1999)], FRET primers (Solinas A et al., 2001. Nucleic Acids Res. 29: E96), AlphaScreen (Beaudet L, et al., Genome Res. 2001, 11(4): 600-8), SNPstream (Bell P A, et al., 2002. Biotechniques. Suppl.: 70-2, 74, 76-7), Multiplex minisequencing (Curcio M, et al., 2002. Electrophoresis. 23: 1467-72), SnaPshot (Turner D, et al., 2002. Hum Immunol. 63: 508-13), MassEXTEND (Cashman J R, et al., 2001. Drug Metab Dispos. 29: 1629-37), GOOD assay (Sauer S, and Gut I G. 2003. Rapid Commun. Mass. Spectrom. 17: 1265-72), Microarray minisequencing (Liljedahl U, et al., 2003. Pharmacogenetics. 13: 7-17), arrayed primer extension (APEX) (Tonisson N, et al., 2000. Clin. Chem. Lab. Med. 38: 165-70), Microarray primer extension (O'Meara D, et al., 2002. Nucleic Acids Res. 30: e75), Tag arrays (Fan J B, et al., 2000. Genome Res. 10: 853-60), Template-directed incorporation (TDI) (Akula N, et al., 2002. Biotechniques. 32: 1072-8), fluorescence polarization (Hsu T M, et al., 2001. Biotechniques. 31: 560, 562, 564-8), Colorimetric oligonucleotide ligation assay (OLA, Nickerson D A, et al., 1990. Proc. Natl. Acad. Sci. USA. 87: 8923-7), Sequence-coded OLA (Gasparini P, et al., 1999. J. Med. Screen. 6: 67-9), Microarray ligation, Ligase chain reaction, Padlock probes, Rolling circle amplification, Invader assay (reviewed in Shi M M. 2001. Enabling large-scale pharmacogenetic studies by high-throughput mutation detection and genotyping technologies. Clin Chem. 47: 164-72), coded microspheres (Rao K V et al., 2003. Nucleic Acids Res. 31: e66), MassArray (Leushner J, Chiu N H, 2000. Mol Diagn. 5: 341-80) and Sequenom Mass Spectrograph technology (e.g., using the iPLEX primer extension and analysis by the MALDI-TOF mass spectrometer; Sequenom, San Diego, Calif.).

The D36Y polymorphs of the VKORC1 polypeptide can be detected by an immunological detection method employed on a protein sample of the individual using an antibody or a fragment thereof which is capable of differentially binding (e.g., by antibody-antigen binding interaction) the polymorphs of the present invention (D36Y). As used herein the phrase “capable of differentially binding” refers to an antibody, which under the experimental conditions employed (as further described hereinunder) is capable of binding to only one polymorph (e.g., VKORC1 Y36) of the protein but not the other polymorph (e.g., VKORC1 D36) or vise versa. Antibodies useful in context of this embodiment of the invention can be prepared using methods of antibody preparation well known to one of ordinary skills in the art, using, for example, synthetic peptides derived from the various domains of the VKORC1 protein for vaccination of antibody producing animals and subsequent isolation of antibodies therefrom. Monoclonal antibodies specific to each of the VKORC1 protein polymorphs can also be prepared as is described, for example, in “Current Protocols in Immunology” Volumes I-III Coligan J. E., Ed. (1994); Stites et al. (Eds), “Basic and Clinical Immunology” (8th Edition), Appleton & Lange, Norwalk, Conn. (1994); Mishell and Shiigi (Eds), “Selected Methods in Cellular Immunology”, W.H. Freeman and Co., New York (1980).

The term “antibody” as used in the present invention includes intact molecules as well as functional fragments thereof, such as Fab, F(ab′)₂, and Fv that are capable of binding to macrophages. These functional antibody fragments are defined as follows: Fab, the fragment which contains a monovalent antigen-binding fragment of an antibody molecule, can be produced by digestion of whole antibody with the enzyme papain to yield an intact light chain and a portion of one heavy chain; Fab′, the fragment of an antibody molecule that can be obtained by treating whole antibody with pepsin, followed by reduction, to yield an intact light chain and a portion of the heavy chain; two Fab′ fragments are obtained per antibody molecule; (Fab′)₂, the fragment of the antibody that can be obtained by treating whole antibody with the enzyme pepsin without subsequent reduction; F(ab′)₂ is a dimer of two Fab′ fragments held together by two disulfide bonds; Fv, defined as a genetically engineered fragment containing the variable region of the light chain and the variable region of the heavy chain expressed as two chains; and single chain antibody (“SCA”), a genetically engineered molecule containing the variable region of the light chain and the variable region of the heavy chain, linked by a suitable polypeptide linker as a genetically fused single chain molecule.

Methods of making these fragments are known in the art. See for example, Harlow and Lane, Antibodies: A Laboratory Manual, Cold Spring Harbor Laboratory, New York, 1988, incorporated herein by reference.

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment.

Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R., Biochem. J., 73: 119-126, 1959. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of V_{H} and V_{L} chains. This association may be noncovalent, as described in Inbar et al., Proc. Nat'l Acad. Sci. USA 69:2659-62, 1972. Alternatively, the variable chains can be linked by an intermolecular disulfide bond or cross-linked by chemicals such as gluteraldehyde. Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow and Filpula, Methods, 2: 97-105, 1991; Bird et al., Science 242:423-426, 1988; Pack et al., Bio/Technology 11:1271-77, 1993; and Ladner et al., U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry, Methods, 2: 106-10, 1991.

As mentioned, the VKORC1 36Y polymorph can be detected in a protein sample of the individual using an immunological detection method. Such methods are fully explained in, for example, “Using Antibodies: A Laboratory Manual” [Ed Harlow, David Lane eds., Cold Spring Harbor Laboratory Press (1999)] and those familiar with the art will be capable of implementing the various techniques summarized hereinbelow as part of the present invention. All of the immunological techniques require antibodies specific to at least one of the VKORC1 D36Y polymorphs. Immunological detection methods suited for use as part of the present invention include, but are not limited to, radio-immunoassay (RIA), enzyme linked immunosorbent assay (ELISA), western blot, immunohistochemical analysis, and fluorescence activated cell sorting (FACS).

Radio-immunoassay (RIA): In one version, this method involves precipitation of the desired substrate, i.e., a protein sample containing the VKORC1 36Y polymorph in this case, with a specific antibody and radiolabelled antibody binding protein (e.g., protein A labeled with I¹²⁵) immobilized on a precipitable carrier such as agarose beads. The number of counts in the precipitated pellet is proportional to the amount of substrate.

In an alternate version of the RIA, a labeled substrate and an unlabelled antibody binding protein are employed. A sample containing an unknown amount of substrate is added in varying amounts. The decrease in precipitated counts from the labeled substrate is proportional to the amount of substrate in the added sample.

Enzyme linked immunosorbent assay (ELISA): This method involves fixation of a sample (e.g., fixed cells or a proteinaceous solution) containing a protein substrate to a surface such as a well of a microtiter plate. A substrate specific antibody (e.g., an antibody capable of binding a protein sample containing the VKORC1 36Y polymorph) coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western blot: This method involves separation of a substrate (a protein sample containing the VKORC136Y polymorph) from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabelled or enzyme linked as described hereinabove. Detection may be by autoradiography, colorimetric reaction or chemiluminescence. This method allows both quantization of an amount of substrate and determination of its identity by a relative position on the membrane which is indicative of a migration distance in the acrylamide gel during electrophoresis.

Immunohistochemical analysis: This method involves detection of a substrate in situ in fixed cells by substrate specific antibodies. The substrate specific antibodies may be enzyme linked or linked to fluorophores. Detection is by microscopy and subjective evaluation. If enzyme linked antibodies are employed, a colorimetric reaction may be required.

Fluorescence activated cell sorting (FACS): This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

The association of the VKORC1 5417T allele (which encodes the VKORC1 Y36 polymorph) with increased predisposition to coumarin resistance provides a tool which can be used to select proper coumarin dosage to individuals in need of coumarin treatment, e.g., doses which on one hand are effective in preventing thrombosis and on the other hand will not subject the individual to increased risks of bleeding and related conditions (e.g., hemorrhages).

In addition, the association of the VKORC1 5417T allele with coumarin resistance can be used to predict the responsiveness of an individual to coumarin treatment.

As used herein the phrase “the responsiveness of an individual to coumarin treatment” refers to the antithrombotic effect of coumarin treatment on the individual, including, but not limited to, prevention of thrombosis and/or related conditions such as stroke, heart failure, ischemia and any thromboembolic manifestation.

It will be appreciated that the reagents utilized by the methods described hereinabove of determining the predisposition to coumarin resistance, determining the responsiveness to coumarin treatment and/or determining if an individual is suitable for genotype analysis of VKORC1 D36Y-related coumarin resistance can form a part of a kit (e.g., a diagnostic kit) and/or an article-of-manufacturing.

Such a kit includes at least one reagent for determining a presence or absence in a homozygous or heterozygous form, of the VKORC1 5417T allele, the VKORC1 Y36 polymorph and/or the VKORC1*1 haplotype. Non-limiting examples of such reagents include an oligonucleotide capable of specifically hybridizing to the VKORC1 5417T allele (e.g., the DNA oligonucleotide set forth by SEQ ID NO:36) using, for example, the ASO hybridization method and/or an antibody of a fragment thereof capable of specifically binding the VKORC1 Y36 polymorph using, for example, the RIA method.

It will be appreciated that such a kit can further include additional reagent(s) suitable for the detection of other polymorphisms which are associated with coumarin resistance and/or sensitivity to thereby increase the predictability power of the kit in determining the predisposition of the individual to coumarin resistance and/or predicting the responsiveness of an individual to coumarin treatment. For example, such reagents can be designed to detect the CYP2C9*1, *2 and/or 3 variants, the VKORC1 861C/A, 5808T/G, 6853G/C, 9041G/A, 5432G/T (A41S), 3673G/A and/or 6484C/T SNPs, the CALU 73G/A (R4Q), 1114G/A and/or 296T/G (S78R) SNPs and/or the EPHX 612T/C SNP. In addition, the kit may further comprise reagents suitable for detecting the VKORC1*1 haplotype, e.g., the wild-type alleles of VKORC1*2 (e.g., the G allele at position 514 of SEQ ID NO:37), VKORC1*3 (e.g., the G allele at position 941 of SEQ ID NO:25), and VKORC1*4 (e.g., the G allele on the + strand (or the C allele on the − strand) at position 256 of SEQ ID NO:38).

According to preferred embodiments the kits further includes packaging material and a notification and/or instructions in or on the packaging material identifying the kits for use in determining if an individual is predisposed to coumarin resistance, determining the responsiveness of the individual to coumarin treatment and/or determining if an individual is suitable for genotype analysis of VKORC1 D36Y-related coumarin resistance.

The kit also includes the appropriate instructions for use and labels indicating FDA approval for use in diagnostics.

As used herein the term “about” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., Ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (Eds.) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., Ed. (1994); “Culture of Animal Cells—A Manual of Basic Technique” by Freshney, Wiley-Liss, N.Y. (1994), Third Edition; “Current Protocols in Immunology” Volumes I-III Coligan J. E., Ed. (1994); Stites et al. (Eds.), “Basic and Clinical Immunology” (8th Edition), Appleton & Lange, Norwalk, Conn. (1994); Mishell and Shiigi (Eds.), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., Ed. (1984); “Nucleic Acid Hybridization” Hames, B. D., and Higgins S. J., Eds. (1985); “Transcription and Translation” Hames, B. D., and Higgins S. J., Eds. (1984); “Animal Cell Culture” Freshney, R. I., Ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Identification of the D36Y VKORC1 Polymorphism in Subjects Requiring High Doses of Warfarin

To elucidate the genetic factors contributing to the variability in warfarin sensitivity, the present inventors have conducted in a group of selected warfarin resistant and sensitive subjects a comprehensive sequence analysis of the VKORC1 and CALU genes along with SNP detection analysis of the *2 and *3 variants of the CYP2C9 gene and the 612T→C SNP of the EPHX1 gene, as follows.

Study subjects, Materials and Experimental Methods

Study subjects: Selected warfarin resistant and warfarin sensitive patients—Subjects were recruited at the Anticoagulation Clinic of the Sheba Medical Center, Israel, which provides anticoagulant therapy for outpatients referred from all hospital facilities. Inclusion criteria were defined as prior (3 month) maintenance of stable therapeutic anticoagulation (±10% stable INR values under constant warfarin doses), normal vitamin K dietary intake and no use of opposing medications. Clinical data included gender, age, ethnic origin, weight, height, indications for warfarin therapy and additional medical conditions. Aliquots of fresh whole blood were drawn for INR determination and DNA extraction. Plasma was separated and aliquots were stored at −20° C. for determination of total plasma warfarin, vitamin K1 and epoxide concentrations. All study participants signed an informed consent form approved by the IRB (Institutional Ethical Review Board) of the Sheba Medical Center. Study subjects included 15 subjects with warfarin resistance (warfarin doses in the range of 80-185 mg/week) and 8 subjects with warfarin sensitivity (warfarin doses in the range of 7-13 mg/week). Warfarin resistant patients were recruited upon indication of stable anticoagulation defined by therapeutic INR values achieved by at least 80 mg warfarin/week in 4 clinic visits, in the absence of any factor known to increase dose requirements. Warfarin sensitive patients were defined by warfarin dose requirements of <13 mg/week to achieve therapeutic INR in 4 visits.

Study subjects: Unselected warfarin-treated subjects—99 subjects warfarin-treated from the Israeli Jewish population were recruited in the course of a previous study [Vecsler M, et al., 2006]. Subjects were maintained with stable (+10%) INR 2.7±0.5 (range 1.9-4.2) by various warfarin doses with a mean of 39.4±7.8 mg/week (range 8-105 mg/week). These patients served as a control group in the present study. Chart data recorded for each patient included gender, age, weight, height, indications for warfarin therapy, additional medical problems and concurrent regularly used medications. A single non-fasting blood sample was taken from each patient approximately 12 hours after the last warfarin dose upon signing of an informed consent approved by the IRB (Institutional Ethical Review Board). An aliquot of fresh whole blood was retained for DNA extraction and genotyping. All study participants signed an informed consent form approved by the IRB of the Sheba Medical Center, Israel.

PCR and sequence analyses—Genomic DNA was extracted from fresh peripheral blood using Puregene commercial kit (Gentra Systems Inc, Minneapolis, Minn.). All PCR reactions for specific amplification of VKORC1, CALU and EPHX1 exonic fragments included 100-300 ng genomic DNA, 0.5 μM of each of the forward (F) and reverse (R) primers, 200 μM dNTPs, 1-2 Units Taq DNA polymerase (Fisher Biotech, Australia), 1.5 mM Mg²⁺ and a compatible reaction buffer. PCR reaction conditions included denaturation for 10 minutes at 95° C. and 30 cycles of: denaturation for 1 minute at 95° C., annealing for 1 minute at 57° C. or 60° C. (as indicated in Table 1, hereinbelow) and elongation for 1 minute at 72° C., followed by a final elongation of 5 minutes at 73° C. Direct sequence analysis was performed in the presence of the same primers using the dye terminator reaction mix and the automated ABI Prism 3100 Avant Genetic Analyzer from Applied Biosystems, Foster City, Calif.). Sequences of VKORC1, CALU and EPHX1 fragments were compared to the sequence in the NCBI bioinformatic database [VKORC1 (gi:46241833, GenBank Accession No. AY587020, SEQ ID NO:25), CALU (gi:6005991, GenBank Accession No. NM_{—}001219, SEQ ID NO:26)] and EPHX1 [GenBank Accession No. NM_{—}000120.2, SEQ ID NO:34]. The presence of CYP2C9*2 and *3 polymorphisms was verified by direct sequence analysis and compared to the NCBI sequence (CYP2C9 gi:13699817; GenBank Accession No. NM_{—}000771; SEQ ID NO:27). The primers used in PCR and sequencing reactions are listed in Table 1, hereinbelow.

TABLE 1

PCR and sequencing primers, and conditions used for genetic
analyses of the VKORC1, CALU, EPHX1 and CYP2C9 genes

| | | PCR | |
|---|---|---|---|
| Primer | | fragment | Annealing |
| index | Primer sequence (5′→3′; SEQ ID NO:) | size | temp. |
| VKORC1 | 5′-CTCCGTGGCTGGTTTTCT (SEQ ID NO:1) | 303 bp | 57° C. |
| exon 1-F | | | |
| VKORC1 | 5′-CCGATCCCAGACTCCAGAAT (SEQ ID NO:2) | | |
| exon 1-R | | | |
| VKORC1 | 5′-TGACATGGAATCCTGACGTG (SEQ ID NO:3) | 361 bp | 57° C. |
| exon 2-F | | | |
| VKORC1 | 5′-GAGCTGACCAAGGGGGAT (SEQ ID NO:4) | | |
| exon 2-R | | | |
| VKORC1 | 5′-AGTGCCTGAAGCCCACAC (SEQ ID NO:5) | 326 bp | 57° C. |
| exon 3-F | | | |
| VKORC1 | 5′-ACCCAGATATGCCCCCTTAG (SEQ ID NO:6) | | |
| exon 3-R | | | |
| CALU | 5′-GTTGGGCGGTGCTTGC (SEQ ID NO:7) | 140 bp | 57° C. |
| exon 1-F | | | |
| CALU | 5′-AAGAAAGCGAATAAAGATGAGGC (SEQ ID | | |
| exon 1-R | NO:8) | | |
| CALU | 5′-TGCCTCCTGAATTAACTGCTTTT (SEQ ID | 289 bp | 57° C. |
| exon 2-F | NO:9) | | |
| CALU | 5′-ACCCCTGAGAGGTGGTACCTTAC (SEQ ID | | |
| exon2-R | NO:10) | | |
| CALU | 5′-GCACACACCTGGCTAAGGCCA (SEQ ID | 314 bp | 57° C. |
| exon3 F | NO:11) | | |
| CALU | 5′-CAAGGTGCCAGCTCCACAA (SEQ ID NO:12) | | |
| exon 3-R | | | |
| CALU | 5′-CCCACCCCACACATTTTCAT (SEQ ID NO:13) | 254 bp | 57° C. |
| exon 4-F | | | |
| CALU | 5′-GAATCCTTCATCTCACCCACCT (SEQ ID | | |
| exon 4-R | NO:14) | | |
| CALU | 5′-CTGGCAAATTCAGAGACCAACTTA (SEQ ID | 213 bp | 57° C. |
| exon 5-F | NO:15) | | |
| CALU | 5′-GTGTTGGGATTACAGGCATGAG (SEQ ID | | |
| exon 5-R | NO:16) | | |
| CALU | 5′-TTAGCATGAGGCAGTTTTGGG (SEQ ID | 301 bp | 57° C. |
| exon 6-F | NO:17) | | |
| CALU | 5′-AATCAAGGATAGAGCGTGTGGG (SEQ ID | | |
| exon 6-R | NO:18) | | |
| CALU | 5′-GCTGTTTTCTTCCCTCTTCGTATG (SEQ ID | 369 bp | 57° C. |
| exon 7-F | NO:19) | | |
| CALU | 5′-CAGTCTCAGTAGCGCAAACAATTT (SEQ ID) | | |
| exon7-R | NO:20) | | |
| CYP2C9* | 5′-TTCAGCAATGGAAAGAAATGG (SEQ ID | 221 bp | 60° C. |
| 2-F | NO:21) | | |
| CYP2C9* | 5′-CCCCTGAAATGTTTCCAAGA (SEQ ID NO:22) | | |
| 2-R | | | |
| CYP2C9* | 5′-GAACGTGTGATTGGCAGAAA (SEQ ID NO:23) | 208 bp | 60° C. |
| 3-F | | | |
| CYP2C9* | 5′-TCGAAAACATGGAGTTGCAG (SEQ ID NO:24) | | |
| 3-R | | | |
| EPHX1-F | 5′-GCTGCTTCCACTATGGCTTC (SEQ ID NO:32) | 234 bp | 60° C. |
| EPHX1-R | 5′-TTGGGTTCTGAATCTCTCCAA (SEQ ID NO:33) | | |

Table 1: Presented are PCR primers and conditions used to amplify the VKORC1 (GenBank Accession No. AY587020; SEQ ID NO:25), the CALU (GenBank Accession No. NM_001219; SEQ ID NO:26), the CYP2C9 (GenBank Accession No. NM_000771; SEQ ID NO:27) and the EPHX1 (GenBank Accession No. NM_000120.2; SEQ ID NO:34) genes.

Nomenclature of the studied genotypes—CYP2C9*1, *2, and *3 genotypes are consistent with the accepted nomenclature (Lee C R, et al., 2002, Pharmacogenetics, 12: 251-63): CYP2C9*1 is R144 and 1359 (430C→T rs1799853 and 1075A→C rs1057910 GenBank Accession No. NM_{—}000771.2), CYP2C9*2 is C144 and 1359 and CYP2C9*3 is R144 and L359 [numbers correspond to GenBank Accession No. NP_{—}000762.2 (SEQ ID NO:30). VKORC1 genotypes are presented according to their position on SEQ ID NO:25 (GenBank Accession No. AY587020) and include the 5440C→T [C43C; Rost S, 2004 (Supra); Geisen C, 2005 (Supra)], the 6484C→T [also known as 1173C→T (Rieder, 2005, Supra) or the VKORC1*2 haplotype) and the 5417G→T (D36Y) SNPs. CALU genotypes are presented according to their position on SEQ ID NO:26 (GenBank Accession No. NM_{—}001219) and include the 1114G→A (SNP rs1043550; also known as 952G→A when numbering refers to the adenosine of the ATG of CALU as nucleotide number “1”), the 73G→A (also known as SNP rs2290228; R4Q; Vecsler et al., 2006) and the novel 296T→G (numbering according to GenBank Accession No. NM_{—}001219; SEQ ID NO:26) resulting in the non-synonymous S78R polymorphism (numbering according to GenBank Accession No. NP_{—}001210; SEQ ID NO:29). EPHX1 genotypes are presented according to their position on SEQ ID NO:34 (mRNA, GenBank Accession No. NM_{—}000120.2) or SEQ ID NO:35 (protein, GenBank Accession No. NP_{—}000111) and include the 612T→C (Y113H).

Data Analyses—Statistical analyses were performed using SPSS software (version 11.0, SPSS Inc.) and GB-STAT software package (version 8.0, Dynamic Microsystems Inc.). Relevant statistical applications are mentioned in the text, odds ratios (OR) are presented with 95% confidence limits, p value of <0.05 indicates statistical significance.

Experimental and Statistical Results

Demographic data of analyzed study subjects—Genetic analysis of the VKORC1, CALU, CYP2C9 and EPHX1 genes was performed in 23 study subjects (e.g., warfarin-treated subjects) which included 11 men and 12 women. The subjects were maintained at INR 2.7±0.5 (range 1.9-4.2) by particularly high (e.g., 80-185 mg/week, i.e., resistant subjects) or low (7-13 mg/week, i.e., sensitive subjects) warfarin doses. Table 2 summarizes the subjects' demographic and clinical data.

TABLE 2
Demographic and clinical data

| Subject | | | | Warfarin dose |
|---|---|---|---|---|
| serial No. | Gender (M/F) | Age (years) | Weight (Kg) | (mg/week) |
| 1 | F | 35 | 65 | 105 |
| 2 | M | 32 | 80 | 110 |
| 3 | F | 43 | 55 | 80 |
| 4 | F | 84 | 51 | 138 |
| 5 | F | 42 | 52 | 185 |
| 6 | M | 60 | 90 | 140 |
| 7 | F | 55 | 72 | 95 |
| 8 | M | 60 | 66 | 115 |
| 9 | F | 34 | 82 | 123 |
| 10 | F | 53 | 70 | 135 |
| 11 | M | 55 | 91 | 80 |
| 12 | F | 48 | 65 | 80 |
| 13 | M | 53 | 90 | 123 |
| 14 | F | 40 | 53 | 95 |
| 15 | F | 31 | 80 | 88 |
| 16 | F | 69 | 68 | 7 |
| 17 | M | 85 | 88 | 7 |
| 18 | M | 60 | 70 | 12 |
| 19 | M | 82 | 71 | 11 |
| 20 | M | 80 | 76 | 13 |
| 21 | M | 55 | 74 | 12 |
| 22 | F | 59 | 65 | 12 |
| 23 | M | 78 | 70 | 10 |

Table 2: The demographic and clinical data of the 23 study subjects are presented.
M = male,
F = female.

As is shown in Table 2, hereinabove, the characteristics of the warfarin resistant subjects (study subjects 1-15) included: male to female ratio 5:10, mean age of 48.3 years (range 31-84), mean weight 70.8 kg (range 51-91) and mean warfarin weekly doses 112.8±29 mg/week (range 80-185). The characteristics of the warfarin sensitive subjects (study subjects 16-23) included: male to female ratio 6:2, mean age of 71 years (range 55-85), mean weight 72.75 kg (range 65-88) and mean warfarin weekly doses 10.5±2.32 mg/week (range 7-13). Warfarin resistant patients were significantly younger compared to the sensitive patients (F=9.79; p=0.002) (Table 2). Body weight, INR values and indications for warfarin therapy were not significantly different between the groups. Warfarin dose requirements were significantly higher in the warfarin resistant group as compared with the warfarin sensitive group. Indications for warfarin therapy included: prosthetic heart valve 51%, atrial fibrillation 18%, deep venous thrombosis (DVT)/pulmonary thromboembolism (PTE) 13%, and others 18%.

TABLE 3
Summary of patient characteristics

| | | | Unselected |
|---|---|---|---|
| | | | warfarin-treated |
| | Warfarin resistant | Warfarin sensitive | patient group |
| Variable | (N = 15) group | (N = 8) group | (N = 99) |
| Age | 48.3 | 71 | 63 |
| (years) | (31-84) | (55-85) | (22-88) |
| Weight | 70.8 ± 23.4 | 72.7 ± 27.7 | 71.4 ± 30.2 |
| (kg) | | | |
| Target INR | 11/15 | 6/8 | 71/99 |
| (2.0-3.0) | | | |
| Target INR | 4/15 | 2/8 | 28/99 |
| (2.5-3.5) | | | |
| Warfarin doses | 112.8 ± 29 | 10.5 ± 2.32 | 39.4 ± 7.8 |
| mg/week | (80-185) | (7-13) | (8-105) |

Genotypes of the VKORC1, CALU, CYP2C9 and EPHX1 genes in selected warfarin sensitive or resistant subjects—Table 4 hereinbelow, summarizes the genotypes of the VKORC1, CALU, CYP2C9 and EPHX1 genes in the 23 study subjects of the present invention.

TABLE 4
Genetic analyses of the CYP2C9, VKORC1, EPHX1 and CALU genes

| Subject | CYP2C9 | VKORC1 | VKORC1 | EPHX1 | CALU | CALU |
|---|---|---|---|---|---|---|
| serial No. | *1/*2/*3 | 6484C→T | 5417G→T | 612T→C | 73G→A | 1114G→A |
| 1 | *1/*1 | C/C | G/G | T/C | G/G | A/A |
| 2 | *1/*1 | C/C | G/G | T/T | G/G | G/G |
| 3 | *1/*1 | C/C | G/G | T/C | G/A | AlA |
| 4 | *1/*1 | C/C | G/T | T/C | G/G | G/G |
| 5 | *1/*1 | C/C | G/T | T/C | G/G | G/G |
| 6 | *1/*1 | C/C | G/T | T/C | A/A | G/G |
| 7 | *1/*1 | C/C | G/G | T/T | G/G | G/G |
| 8 | *1/*1 | C/C | G/G | T/C | G/G | G/A |
| 9 | *1/*1 | C/C | G/T | T/T | G/G | G/G |
| 10 | *1/*2 | C/C | G/T | T/T | G/G | G/A |
| 11 | *1/*3 | C/T | G/T | T/T | G/A | G/A |
| 12 | *1/*1 | C/T | G/G (a) | T/T | A/A | A/A |
| 13 | *1/*1 | C/T | G/T | T/T | G/G | G/A |
| 14 | *1/*1 | C/C | G/G | T/T | G/G | G/A |
| 15 | *1/*1 | C/C | G/G | T/C | G/G | G/A |
| 16 | *3/*3 | C/C | G/G | T/C | G/A | G/G |
| 17 | *3/*3 | C/T | G/G | T/T | G/G | A/A |
| 18 | *1/*1 | T/T | G/G | T/C | G/G | G/G |
| 19 | *1/*1 | T/T | G/G | T/T | G/G | G/A |
| 20 | *1/*3 | T/T | G/G | T/T | G/A | G/A (b) |
| 21 | *1/*3 | C/T | G/G | T/C | G/A | G/A |
| 22 | | T/T | G/G | T/C | G/A | G/A |
| 23 | *1/*2 | C/T | G/G | T/T | G/A | A/A |

Table 4: Shown are genotypes of the CYP2C9, VKORC1, EPHX1 and CALU genes in warfarin resistant (subjects 1-15) and warfarin sensitive (subjects 16-23) subjects. The nomenclature of the genotypes is as described under “Material and Experimental Methods” hereinabove.
(a) indicates a rare VKORC1 5440C→T (C43C) polymorphism detected in subject No. 12;
(b) indicates a novel CALU S78R polymorphism detected in subject No. 20.
The VKORC1 5417G/T and the CALU 73G/A genotypes are associated with warfarin resistance and the VKORC1 6484C/T, 6484T/T, CYP2C9*1/*3 and CYP2C9*3/*3 are associated with warfarin sensitivity.

Identification of the VKORC1 D36Y polymorphism in warfarin resistant subjects—In the warfarin resistant group (subjects 1-15, Table 4 hereinabove), the non-synonymous D36Y polymorphism in the VKORC1 gene was found in an unexpectedly high frequency. This missense mutation, designated VKORC1 D36Y (5417G→T according to SEQ ID NO:25; GenBank Accession No. AY587020) was present in 7 out 15 (46.6%) of the resistant warfarin subjects and was absent in the warfarin sensitive subjects [subjects Nos. 16-23, Table 4 hereinabove; (Fishers exact p=0.026)].

Detection of a previously known polymorphism in the VKORC1 gene—The VKORC16484C→T polymorphism in intron 1, which is known to be associated with low warfarin dose requirement [Rieder M J, 2005 (Supra), Geisen, 2005 (Supra)], was also found in the present study to be more prevalent in the warfarin sensitive group (11 out of 16 chromosomes in subjects Nos. 16-23, of them 3 were heterozygotes and 4 homozygotes for the 6484T allele) than in the warfarin resistant group (3 out of 30 chromosomes in subjects Nos. 1-15, Table 4). Similarly, the CYP2C9*3 allele was more prevalent among warfarin sensitive subjects (6 out of 16 chromosomes in subjects Nos. 16-23) than among warfarin resistant subjects (1 out of 30 chromosomes in subjects Nos. 1-15, Table 4).

The VKORC1 Y36 variant prevails the effects of VKORC1 6484T and CYP2C9*3 variants—The results shown in Table 4 hereinabove indicate that the effect of the VKORC1 Y36 variant [i.e., a tyrosine residue-containing polymorph at position 36 of SEQ ID NO:28 (GenBank Accession No. AA583106)] contributing to higher warfarin doses prevails the opposing effects of VKORC1 6484T and CYP2C9*3 related to lower warfarin doses. In the resistant group, two subjects were identified with VKORC1 6484T/5417T genotypes (subject No. 13, Table 4) and VKORC1 6484T/5417T and CYP2C9*3 genotypes (subject No. 11, Table 4), who were optimized at 123 mg and 80 mg warfarin per week, respectively. In addition, in another subject who received a warfarin dose of 80 mg/week (subject No. 12, Tables 2 and 4), the VKORC1 6484T allele was present along with a rare synonymous VKORC1 5440C→T (C43C) polymorphism [Geisen, 2005 (Supra)].

Genotypes of the CALU gene in warfarin resistant and sensitive subjects—Sequence analysis of the seven CALU exons including at least 50 nucleotides of the intron-exon boundaries, yielded the R4Q polymorphism and 1114G→A polymorphism in the untranslated seventh exon [SNP rs1043550]. In a recent study performed by the present inventors [Vecsler M, et al., 2006] homozygotes of the R4Q polymorphism (i.e., protein polymorphs having Glutamine (Q) residue at position 4 of SEQ ID NO:29) have been related to warfarin resistance. In the present study, two warfarin resistant subjects with the CALU 73A/A genotype (4Q) were detected (subjects Nos. 2 and 12, Table 4, hereinabove), while no CALU 73A/A genotype was detected among the sensitive subjects. Homo- and heterozygote carriers of the CALU 1114A allele were present in both the warfarin resistant and sensitive groups. One warfarin sensitive subject (subject No. 20, Table 4) was a carrier of CALU 1114A allele and yet another novel polymorphism S78R in CALU exon 3.

Genotypes of the EPHX1 gene in warfarin resistant and sensitive subjects—In a previous study [Loebstein R, et al., 2005 (Supra)], which involved 100 patients undergoing therapeutic anticoagulation, the present inventors have demonstrated that beyond the known pharmacokinetic effects of advanced age and CYP2C9 genotypes on warfarin dose requirements, the EPHX1 612C allele confers a pharmacodynamic effect that is associated with increased warfarin dose requirements. These findings were only demonstrated for the 2 upper quartiles of the warfarin dose distribution (>50 mg/week), reflecting the nonlinear dose-effect relationship of warfarin. As is shown in Table 4, hereinabove, the EPHX1 612C allele was present in 7 out of 30 chromosomes of the warfarin resistance subjects and in 4 out of 16 chromosomes of the warfarin sensitive subjects.

Altogether, these finding demonstrate that the unexpectedly high association of the VKORC1 D36Y polymorphism (VKORC1 5417G→T) with warfarin resistance, especially in warfarin-treated subjects requiring an average weekly dose of 112.8 mg warfarin (e.g., 16 mg/day). Thus, these findings suggest the use of the VKORC1 5417G→T polymorphism in determining the predisposition to coumarin (e.g., warfarin) resistance and/or predicting the responsiveness of a subject to coumarin treatment.

### Example 2

### The VKORC1 D36Y Polymorphism is Associated with Warfarin Resistance in an Unselected Group of Warfarin-Treated Subjects

To substantiate the association of the D36Y polymorphism in the VKORC1 gene with coumarin resistance, the present inventors have conducted an RFLP analysis of the VKORC1 5417G→T SNP in a group of 99 unselected subjects who were treated with various warfarin doses and 600 anonymous DNA samples from various ethnic groups, as follows.

Materials and Experimental Methods

Study subjects: Unselected warfarin-treated group as described in Example 1, hereinabove.

Population frequency of VKORC1 Asp36Tyr—Frequency of the VKORC1 Asp36Tyr polymorphism was determined in four distinct ethnic groups from the general Israeli Jewish population (Ashkenazi, Yemenite, North-African and Ethiopian origin). DNA samples were retrieved from the Prenatal Genetic Screening Program depository at the Genetics Institute, Sheba Medical Center. This program is directed at prenatal diagnosis of inherited monogenic disorders (cystic fibrosis, Tay Sachs, etc.) unrelated to any cardiovascular diseases. Recordings include information on the ethnic origin of both parental sides in the past two generations. Relying on these data, retrieved anonymously, were constructed four ethnic groups each consisting of 100 DNA samples (200 chromosomes).

Data analysis—Data are presented as mean±SD. Means were compared across groups by ANOVA. Chi square and Fisher exact tests were used to compare the frequencies of CYP2C9 and VKORC1 haplotypes, the VKORC1 Tyr36Asp, and CALU and EPHX1 polymorphisms in the warfarin resistant and sensitive groups, and in the unselected, warfarin-treated group of subjects. Multiple linear regressions were used to determine the independent effects of the genetic variants, and age and weight on warfarin dose requirements in the control group. Logistic regression was used to determine the relative effect of the genetic variants, age and weight on warfarin dose requirements in the control group. In these analyses, patients were grouped as: above/below 20 mg/week or above/below 50 mg/week or above/below 70 mg/week. These cut-offs were defined by the 25^{th}, 50^{th} (median) and 75^{th} dose percentiles in the controls. Odds ratios are presented as OR (95% C.L.). Analyses were carried out using GB-STAT statistical software package (Dynamic Microsystems Inc, Silver Spring Md.).

RFLP analysis for the VKORC1 D36Y polymorphism—Population screening for the presence of VKORC1 D36Y polymorphism (5417G→T according to SEQ ID NO:25) was performed using RFLP recognized by the Rsa1 restriction endonuclease. PCR was performed using the 5′-CTCCGTGGCTGGTTTTCT (SEQ ID NO:1) and 5′-CCGATCCCAGACTCCAGAAT (SEQ ID NO:2) PCR primers (listed in Table 1) and the resultant 303 bp PCR product was subjected to RsaI restriction analysis. Digestion reactions were carried out in a 15 μl reaction volume including 2.5 μl PCR product, 10 Units Rsa1 (Fermentas UAB, Lithuania) and 1.5 μl compatible buffer, and incubated overnight at 37° C. Digestion products were analyzed on 2% agarose gel containing ethidium bromide in TBE buffer. Digestion of a PCR product containing the 5417T allele with RsaI resulted in two fragments of 155 and 148 bp (migrating as a ˜150 bp band on the agarose gel). Heterozygotes to the 5417G/T polymorphism exhibited 2 fragments of 300 bp and ˜150 bp. Homozygote to the 5417G allele exhibited only the 303 bp band.

Data Analysis—performed as described in Example 1, hereinabove.

Experimental Results

The D36Y polymorphism is more prevalent among high dose warfarin-treated subjects—The presence of carriers of D36Y polymorphism was verified in 99 warfarin-treated subjects from the Israeli Jewish population that were maintained with stable INR 2.7±0.5 (range 1.9-4.2) by various warfarin doses with a mean of 39.4±7.8 mg/week (range 8-105 mg/week). As is shown in Table 5, hereinbelow, the overall frequency of the D36Y polymorphism in this group of warfarin-treated subjects was 8/198 chromosomes (8 heterozygote subjects). These 8 Asp36Tyr carriers were all in the upper quartile of warfarin dose requirements (70-105 mg/week) compared to the other 91 patients lacking this polymorphism (8-70 mg/week). Thus, carriers of Asp36Tyr in the control group (8/99) required significantly higher warfarin doses of an average of 80.9±10.1 mg/week compared to an average of 42.7±7.5 mg/week in non-carriers (Anova test F=9.79, p=0.002). Asp36Tyr was significantly associated with doses of >70 mg/week (OR=13.0 [95% C.I. 1.3-124.2]).

These results demonstrate that even in the unselected group of warfarin-treated subjects, the presence of the D36Y polymorphism is associated with high dose requirement of warfarin. Thus, these results are consistent with the findings described in Example 1, hereinabove, of high prevalence (7/15 subjects) of the D36Y polymorphism in warfarin resistant subjects (i.e., subjects treated with 80-185 mg warfarin/week; Table 4, hereinabove). It should be noted that the representation of D36Y carriers in the exclusive group of selected resistant subjects (which were maintained on relatively high warfarin doses such as 112.8 mg warfarin per week (e.g., 16 mg/day) was significantly higher than in the unselected group of 99 warfarin-treated subjects (7 out of 15 as compared to 8 out of 99, yielding χ²=13.94, p<0.001). These results may suggest that the D36Y polymorphism is a major contributor to relatively high resistance to warfarin treatment in subjects who require relatively high doses of warfarin.

TABLE 5
Distribution of the VKORC1 D36Y polymorphism in
99 warfarin-treated subjects

Warfarin dose percentile

| | 25% | 50% | 75% | >75% |
|---|---|---|---|---|
| Dose (mg/week) | 22.5 | 35 | 52.5 | >52.5 |
| No. of subjects | 22 | 26 | 27 | 25 |
| No. of D36Y carriers | 0 | 0 | 0 | 8 |

Table 5: The frequency of the VKORC1 D36Y polymorphism in an unselected warfarin-treated subjects is presented according to the warfarin dose percentile.
The upper quartile (>75 percentile) is ≧70 mg/week.

Altogether, these results demonstrate, for the first time, the unexpectedly high association of the VKORC1 5417T allele (i.e., the Y36 polymorph) with warfarin resistance and/or requirement of high doses of warfarin for the prevention of thromboembolism.

The D36Y polymorphism (genotype 5417G→T) is more prevalent among Ethiopian Jewish subjects than among other Jewish populations—The prevalence of the D36Y polymorphism was studied in various ethnic groups of the Jewish population using anonymous DNA samples with distinct characteristics with respect to the ethic origin from the paternal and maternal lines in the last two generations. Table 6, hereinbelow, demonstrates population frequencies of D36Y polymorphism in 4 ethnic groups (Jewish Ashkenazi, Yemenite, Moroccan (North Africa) and Ethiopian) each consisting of 100 individuals (i.e., 200 chromosomes) selected for common origin on both parental sides. The results show that the D36Y polymorphism is significantly over-represented in the Ethiopian group (15%), which included 25 heterozygous and 3 homozygous individuals. The D36Y polymorphism was also found, however to a lesser extent, in the Ashkenazi Jewish group (4%) and in the Moroccan (North Africa) and Yemenite Jewish (0.5% in each group).

TABLE 6
Frequency of VKORC1 D36Y polymorphism
in various ethnic groups

| | No. of | Frequency of |
|---|---|---|
| Ethnic group | chromosomes | D36Y allele |
| Ashkenazi Jewish | 200 | 4% |
| Yemenite Jewish | 200 | 0.5% |
| Moroccan Jewish | 200 | 0.5% |
| Ethiopian Jewish | 200 | 15% |

Table 6: The frequency of the VKORC1 D36Y polymorphism in various ethnic groups is presented.
Note the high prevalence of the D36Y polymorphism among the Ethiopian Jewish ethnic group.

Altogether, the high frequency of the D36Y polymorphism, which is associated with warfarin resistance, among the Ethiopian Jewish population, may suggest that individuals of this ethnic group exhibit increased predisposition risk to warfarin resistance as compared to the other Jewish ethnic groups.

### Example 3

### VKORC1 Haplotypes

Materials and Experimental Methods

VKORC1 haplotyping—VKORC1 haplotypes were determined according to the presence of the following tag-SNPs: for VKORC1*2, the G→C substitution at position 514 of SEQ ID NO:37 (SNP rs8050894; also referred to as 6853G→C in Table 1 of Geisen et al., 2005; Thromb Haemost. 94:773-9); for VKORC1*3, the G→A substitution at position 941 of SEQ ID NO:25 (rs7294; also referred to as 9041G→A in Table 1 of Geisen et al., 2005; Thromb Haemost. 94:773-9), for VKORC1*4, the G→A substitution at position 256 of SEQ ID NO:38 (rs17708472; also referred to as 6009C→T in Table 1 of Geisen et al., 2005; Thromb Haemost. 94:773-9); and lack of these was considered as VKORC1*1. In addition, 5417G→T of SEQ ID NO:25, representing the Asp36Tyr polymorphism was included.

Determination of haplotypes using Sequenom Mass Spectrograph technology—PCR reactions were carried out in 5 μl volumes containing 5 ng genomic DNA, 0.1 U high-fidelity Taq polymerase (HotStar, QIAGEN, USA), 2.5 pmol of each PCR primer, 2.5 μmole dNTPs and cycling at 95° C.-15 minutes; 45 cycles of 95° C.-20 seconds, 56° C.-30 seconds, 72° C.-30 seconds. Unincorporated dNTPs were deactivated using 0.3 U shrimp alkaline phosphatase (USB, USA) at 37° C.-20 minutes and at 85° C.-5 minutes. Primer extension was carried out using iPLEX reaction (Sequenom, San Diego, Calif.) including 5.4 pmol of each primer, 50 μmole ddNTP, 0.5 U Thermosequenase (Sequenom) and cycling of 94° C.-2 minutes; 40 cycles of 94° C.-5 seconds, 50° C.-5 seconds, 72° C.-5 seconds. Primer extension products were desalted using SpectroCLEAN cation exchange resin (Sequenom), 15 μl of the products were loaded by Samsung Nanodispenser onto the SpectroCHIP microarray (both by Sequenom) and analyzed using a Bruker Biflex MALDI-TOF mass spectrometer (SpectroREADER, Sequenom). The spectra were processed using SpectroTYPER (Sequenom), assays in which 85% of all genotyping calls were obtained were considered successful.

Tables 7 and 8 hereinbelow, provide SNPs information (Table 7) and primer sequences (Table 8) of SNPs used to determine haplotypes in the present study by Sequenom analysis.

TABLE 7

| | | SNP | | |
|---|---|---|---|---|
| | GenBank | Nucleotide | Ref | |
| SNP ID | Accession No. | Site | Sequence | SNP sequence |
| CYP2C9*2 | NM_000771.2 | 430C→T | rs1799853 | GTTTCTCCCT CATGACGCTG |
| R144C | | | | CGGAATTTTG GGATGGGGAA |
| | | | | GAGGAGCATT GAGGAC [C/T] |
| | | | | GTGTTCAAGA GGAAGCCCGC |
| | | | | TGCCTTGTGG AGGAGTTGAG |
| | | | | AAAAACCAAG GGTGGGTGAC |
| | | | | (SEQ ID NO: 39) |
| CYP2C9*3 | NM_000771.2 | 1075A→C | rs1057910 | AGACAGGAGC CACATGCCCT |
| 1359L | | | | ACACAGATGC TGTGGTGCAC |
| | | | | GAGGTCCAGA GATAC [A/C] |
| | | | | TTGACCTTCT CCCCACCAGC |
| | | | | CTGCCCCATG CAGTGACCTG |
| | | | | TGACATTAAA TTCAGAAACT |
| | | | | (SEQ ID NO: 40) |
| VKORC1*2 | AY587020 | 6484C→T | rs9934438 | TGATTTCCAA GAAGCCACCT |
| | | | tag | GGGCTATCCT CTGTTCCCCG |
| | | | minor | ACCTCCCATC CTAGTCCAAG [A/G] |
| | | | antisense | GTCGATGATC TCCTGGCACC |
| | | | | GGGCACCTTT GGCCACGTCA |
| | | | | GGATTCCATG TCACTGACCC |
| | | | | (SEQ ID NO: 41) |
| VKORC1*3 | AY587020 | 9041G→A | rs7294 tag | GGGTCCCTAG AAGGCCCTAG |
| | | | minor | ATGTGGGGCT TCTAGATTAC |
| | | | | CCCCTCCTCC TGCCATACCC [A/G] |
| | | | | CACATGACAA TGGACCAAAT |
| | | | | GTGCCACACG CTCGCTCTTT |
| | | | | TTTACACCCA GTGCCTCTGA |
| | | | | (SEQ ID NO: 42) |
| VKORC1*4 | AY587020 | 6009C→T | rs17708472 | TACAGGAGTG GGCCACCGCG |
| | | | tag minor | CCCGGCCCTT AAGTAATTCT |
| | | | antisense | TAAAATGGCA AGGCT [A/G] |
| | | | | GTATAACGGT TCACTCGGTT |
| | | | | TTGCATCAGA GACTGGGAGT |
| | | | | CGGGGGCAGA TTATCTTTGC |
| | | | | (SEQ ID NO: 43) |
| VKORC1 | AY587020 | 5417G→T | NEW | TGACGGGCTT AGTGCTCTCG |
| D36Y | | | | CTCTACGCGC TGCACGTGAA |
| | | | | GGCGGCGCGC GCCCGG [G/T] ACC |
| | | | | GGGATTACCG CGCGCTCTGC |
| | | | | GACGTGGGCA CCGCCATCAG |
| | | | | CTGTTCGCGC GTCTTCTCCT |
| | | | | (SEQ ID NO: 44) |
| CALU R4Q | NM_001219 | 73G→A | rs2290228 | TTTTCTGTGG GTTTGCTCAA |
| | | | antisense | GGCAAAGGCT GTGCACAGGG |
| | | | | ACAGGCACAT AAGAAACTGT [C/T] |
| | | | | GCAGGTCCAT GATAATTAGA |
| | | | | TCTTGAAGAA AATGAAAAGC |
| | | | | AGTTAATTCA GGAGGCAGTA |
| | | | | (SEQ ID NO: 45) |
| CALU | NM_001219 | 358T→G | new | TAGATGGCGA CAAGGACGGG |
| S78R | | | | TTTGTCACTG TGGATGAGCT |
| | | | | CAAAGACTGG ATTAAAT [T/G] TG |
| | | | | CACAAAAGCG CTGGATTTAC |
| | | | | GAGGATGTAG AGCGACAGTG |
| | | | | GAAGGGGCAT GACCTCAATG |
| | | | | (SEQ ID NO: 46) |
| CALU | NM_001219 | 1114G→A | rs1043550 | GTTGGCAGCC AGGCCACAGA |
| 3′UTR | | | minor | TTTTGGGGAG GCCTTAGTAC |
| | | | | GGCATGAT GAGTTCTGAG CT [A/G] |
| | | | | CGGAGGAACC CT CATTTCCTCA |
| | | | | AAAGTAATTT ATTTTTACAG |
| | | | | CTTCTGGTTT |
| | | | | (SEQ ID NO: 47) |
| EPHX1 | NM_000120.2 | 612T→C | rs1051740 | GTCATCTCCT ACTGGCGGAA |
| Y113H | | | minor | TGAATTTGAC TGGAAGAAGC |
| | | | | AGGTGGAGAT TCTCAACAGA [C/T] |
| | | | | ACCCTCACTT CAAGACTAAG |
| | | | | ATTGAAGGTA TGTTTGCAAA |
| | | | | ACGCCAGCCA GAGAGGGATG |
| | | | | (SEQ ID NO: 48) |
| GCCX | U65896.1 | 8762G→A | | GCCAGCAGCC CTCTCTTCTG |
| Q325R | | | | CTCCCCTGAG TGGCCTCGGA |
| | | | | AGCTGGTGTC CTACTGCCCC C [G/A] |
| | | | | AAGGTTGC AACAACTGTT |
| | | | | GCCCCTCAAG GCAGCCCCTC |
| | | | | AGCCCAGTGT TTCCTGTGTG |
| | | | | (SEQ ID NO: 49) |

TABLE 8

| | |
|---|---|
| SNP ID | Primers' Sequences |

| | | |
|---|---|---|
| CYP2C9*2 Forward | ACGTTGGATGATGACGCTGCGGAATTTTGG | (SEQ ID NO: 50) |
| VKORC1*4 Forward | ACGTTGGATGCCCAGTCTCTGATGCAAAAC | (SEQ ID NO: 51) |
| EPHX1 Y113H Forward | ACGTTGGATGTTGACTGGAAGAAGCAGGTG | (SEQ ID NO: 52) |
| CYP2C9*3 Forward | ACGTTGGATGTGTCACAGGTCACTGCATGG | (SEQ ID NO: 53) |
| CALU 3UTR Forward | ACGTTGGATGTACTTTTGAGGAAATGAGGG | (SEQ ID NO: 54) |
| VKORC1*2 Forward | ACGTTGGATGTGACATGGAATCCTGACGTG | (SEQ ID NO: 55) |
| VKORC1*3 Forward | ACGTTGGATGAAAAAAGAGCGAGCGTGTGG | (SEQ ID NO: 56) |
| GCCX Q325R Forward | ACGTTGGATGTACACACAGGAAACACTGGG | (SEQ ID NO: 57) |
| CALU S78R Forward | ACGTTGGATGCACTGTGGATGAGCTCAAAG | (SEQ ID NO: 58) |
| CALU R4Q Forward | ACGTTGGATGCTGCCTCCTGAATTAACTGC | (SEQ ID NO: 59) |
| VKORC1 Asp36Tyr | ACGTTGGATGAAGACGCGCGAACAGCTGAT | (SEQ ID NO: 60) |
| Forward | | |
| CYP2C9*2 Reverse | ACGTTGGATGTCAACTCCTCCACAAGGCAG | (SEQ ID NO: 61) |
| VKORC1*4 Reverse | ACGTTGGATGGCCCGGCCCTTAAGTAATTC | (SEQ ID NO: 62) |
| EPHX1 Y113H Reverse | ACGTTGGATGTGGCGTTTTGCAAACATACC | (SEQ ID NO: 63) |
| CYP2C9*3 Reverse | ACGTTGGATGCTACACAGATGCTGTGGTGC | (SEQ ID NO: 64) |
| CALU 3UTR Reverse | ACGTTGGATGCAGCCAGGCCACAGATTTTG | (SEQ ID NO: 65) |
| VKORC1*2 Reverse | ACGTTGGATGACCTGGGCTATCCTCTGTTC | (SEQ ID NO: 66) |
| VKORC1*3 Reverse | ACGTTGGATGTTCTAGATTACCCCCTCCTC | (SEQ ID NO: 67) |
| GCCX Q325R Reverse | ACGTTGGATGTCGGAAGCTGGTGTCCTACT | (SEQ ID NO: 68) |
| CALU S78R Reverse | ACGTTGGATGTTCCACTGTCGCTCTACATC | (SEQ ID NO: 69) |
| CALU R4Q Reverse | ACGTTGGATGACAGGGACAGGCACATAAG | (SEQ ID NO: 70) |
| VKORC1 Asp36Tyr | ACGTTGGATGTCTACGCGCTGCACGTGAA | (SEQ ID NO: 71) |
| Reverse | | |
| CYP2C9*2 Extension | AGAGGAGCATTGAGGAC | (SEQ ID NO: 72) |
| VKORC1*4 Extension | CCGAGTGAACCGTTATAC | (SEQ ID NO: 73) |
| EPHX1 Y113H Extension | GGTGGAGATTCTCAACAGA | (SEQ ID NO: 74) |
| CYP2C9*3 Extension | GCTGGTGGGGAGAAGGTCAA | (SEQ ID NO: 75) |
| CALU 3UTR Extension | GAGGAAATGAGGGTTCCTCCG | (SEQ ID NO: 76) |
| VKORC1*2 Extension | CCCGGTGCCAGGAGATCATCGAC | (SEQ ID NO: 77) |
| VKORC1*3 Extension | TGGTCCATTGTCATGTG | (SEQ ID NO: 78) |
| GCCX Q325R Extension | CAACAGTTGTTGCAACCTT | (SEQ ID NO: 79) |
| CALU S78R Extension | GCTCAAAGACTGGATTAAAT | (SEQ ID NO: 80) |
| CALU R4Q Extension | ATCTAATTATCATGGACCTGC | (SEQ ID NO: 81) |
| VKORC1 Asp36Tyr | CAGAGCGCGCGGTAATCCCGGT | (SEQ ID NO: 82) |
| Extension | | |

Primers Used for VKORC1 Tag-SNP:

For VKORC1*2 (G→C substitution at position 514 of SEQ ID NO:37) the primers set forth by SEQ ID NOs:55, 66 and 77 were used.

For VKORC1*3 (G→A substitution at position 941 of SEQ ID NO:25) the primers set forth by SEQ ID NOs:56, 67 and 78 were used.

For VKORC1*4 (G→A substitution at position 256 of SEQ ID NO:38) the primers set forth by SEQ ID NOs:51, 62 and 73 were used.

Genotyping of the VKORC1 Asp36Tyr polymorphism—was performed using the primers set forth by SEQ ID NOs:60, 71 and 82.

Experimental Results

Genotyping data of the Asp36Tyr polymorphism with respect to known VKORC1 haplotypes—Table 9, hereinbelow, demonstrates the co-presence of the Asp36Tyr polymorphism on known VKORC1 haplotypes as well as on genotypes and haplotypes of other candidate risk genes.

TABLE 9
Genotypes in the warfarin resistant and sensitive patients' groups

| Subject | | Dose | CYP2C9 | VKORC1 | VKORC1 | CALU | EPHX1 |
|---|---|---|---|---|---|---|---|
| ID NO: | Gender | mg/week | haplotypes | haplotypes | Asp36Tyr | Arg4GLu | Tyr113His |
| 1 | F | 105 | *1/*1 | *3/*4 | Wt/Wt | Wt/Wt | Wt/Mut |
| 2 | M | 110 | *1/*1 | *3/*3 | Wt/Wt | Wt/Wt | Wt/Wt |
| 3 | F | 80 | *1/*1 | *2/*3 | Wt/Wt | Wt/Mut | Wt/Mut |
| 4 | F | 138 | *1/*1 | *1/*4 | Wt/Mut | Wt/Wt | Wt/Mut |
| 5 | F | 185 | *1/*1 | *1/*3 | Wt/Mut | Wt/Wt | Wt/Mut |
| 6 | M | 140 | *1/*1 | *1/*4 | Wt/Mut | Mut/Mut | Wt/Mut |
| 7 | F | 95 | *1/*1 | *4/*4 | Wt/Wt | Wt/Wt | Wt/Wt |
| 8 | M | 115 | *1/*1 | *3/*4 | Wt/Wt | Wt/Wt | Wt/Mut |
| 9 | F | 123 | *1/*1 | *1/*3 | Wt/Mut | Wt/Wt | Wt/Wt |
| 10 | F | 135 | *1/*2 | *1/*4 | Wt/Mut | Wt/Wt | Wt/Wt |
| 11 | M | 80 | *1/*3 | *1/*2 | Wt/Mut | Wt/Mut | Wt/Wt |
| 12 | F | 80 | *1/*1 | *2/*3 | Wt/Wt | Mut/Mut | Wt/Wt |
| 13 | M | 123 | *1/*1 | *1/*2 | Wt/Mut | Wt/Wt | Wt/Wt |
| 14 | F | 95 | *1/*1 | *3/*4 | Wt/Wt | Wt/Wt | Wt/Wt |
| 15 | F | 88 | *1/*1 | *3/*3 | Wt/Wt | Wt/Wt | Wt/Mut |
| 16 | F | 7 | *3/*3 | *1/*3 | Wt/Wt | Wt/Mut | Wt/Mut |
| 17 | M | 7 | *3/*3 | *2/*4 | Wt/Wt | Wt/Wt | Wt/Wt |
| 18 | M | 12 | *1/*3 | *2/*2 | Wt/Wt | Wt/Mut | Wt/Mut |
| 19 | M | 11 | *1/*1 | *2/*2 | Wt/Wt | Wt/Wt | Wt/Wt |
| 20 | M | 13 | *1/*3 | *2/*2 | Wt/Wt | Wt/Mut | Wt/Wt |
| 21 | M | 12 | *1/*1 | *2/*2 | Wt/Wt | Wt/Wt | Wt/Mut |
| 22 | F | 12 | *1/*3 | *2/*2 | Wt/Wt | Wt/Mut | Wt/Mut |
| 23 | M | 10 | *1/*2 | *2/*3 | Wt/Wt | Wt/Mut | Wt/Wt |

Table 9: Patients' numbers 1-15 are Warfarin resistant, patients' numbers 16-23 are Warfarin sensitive. CYP2C9*1, *2 and *3 are consistent with the conventional nomenclature and are described in Example 1, hereinabove, VKORC1*2, *3 and *4 correspond to the presence of the tag-SNPs described under Materials and Experimental Methods, hereinabove. Mut are heterozygotes for VKORC1 5417G→T (Asp36Tyr) or CALU 73G→A (R4Q; Arg4Glu) or EPHX1 612T→C (Tyr113His), and Mut/Mut are homozygotes.

Determinants of Warfarin Dose Categories

Warfarin resistance (>70 mg/week)—As described in Example 1, hereinabove, the non-synonymous VKORC1 5417G→T (Asp36Tyr) polymorphism was identified in 7/15 resistant patients but not in the 8 sensitive patients (p=0.026). As further described in Example 2, hereinabove, in the unselected group of warfarin treated patients the Asp36Tyr was present in 8/99 patients (all in the upper dose quartile) requiring 80.9±10.1 mg/week, compared to the non-carriers (n=91) requiring 42.7±7.5 mg/week (F=9.28; p=0.0001). Categorical analysis of warfarin dose requirements (Table 10, hereinbelow) revealed that the Asp36Tyr polymorphism was significantly associated with the highest warfarin dose category (>70 mg/week) (OR=13.0 [1.3−124.2]) with no effect on the intermediate (20-70 mg/week) and low (<20 mg/week) dose categories (Table 10, hereinbelow). Patients with Asp36Tyr and other dose-reducing markers (CYP2C9*2, *3, and VKORC1*2) still required high warfarin doses (See for example, subjects Nos. 10, 11 and 13 in Table 9, hereinabove). Co-presence of Asp36Tyr with VKORC1*3 or *4 haplotypes manifested higher warfarin doses than VKORC1*3 or *4 alone (Table 9 and data not shown). In all cases, the new VKORC1 Asp36Tyr polymorphism was co-present with the tag-SNP of the wild type VKORC1*1 haplotype.

The warfarin resistant group included two homozygotes for the CALU Arg4Glu polymorphism and two heterozygotes (4/15) compared to 5/8 in the sensitive group (Table 9). One of the homozygotes requiring 140 mg/week (previously described) was also a carrier of Asp36Tyr. The second requiring 80 mg/week had no other dose incrementing genotypes. In the control group, CALU Arg4Glu was found in 38/99, all heterozygotes (NS). The distribution of the EPHX1 612T→C (Tyr113His) polymorphism did not differ significantly between the resistant (7/15) and sensitive (4/8) (Table 9), and control (42/99) groups.

TABLE 10
Association of dose requirements (OR) with
constitutional and genetic determinants

| Variables | <20 mg/week | ≧70 mg/week |
|---|---|---|
| Age | 1.05 (1.02-1.14) | 0.93 (0.87-0.98) |
| | 0.04 | 0.04 |
| Weight | NS | NS |
| CYP2C9*2/*3 | 2.4 (1.3-4.6) | NS |
| | 0.03 | |
| VKORC1*2 | NS | NS |
| VKORC1 Asp36Tyr | NS | 13.0 (1.3-124.2) |
| | | 0.02 |

Table 10: Association of dose requirements (Odds Ratio; OR) with constitutional and genetic determinants.
Warfarin dose requirements were categorized as high (≧70 mg/week) and low (<20 mg/week) and were analyzed by the logistic regression.
Provided are Odds Ratio with approximate confidence intervals, and p values.
NS = non-significance.

Warfarin sensitivity (<20 mg/week)—The dose-reducing CYP2C9*2 and *3 alleles were over-represented in the warfarin sensitive (8 of total 16 alleles) compared to the warfarin resistant (2/30) group (Fisher exact p=0.001). Similarly, the tag-SNP of the VKORC1*2 haplotype was more prevalent in the warfarin sensitive (12/16 alleles) compared to the resistant (4/30) group (Fisher exact p<0.0001). Categorical analysis of the control group showed that warfarin sensitivity (<20 mg/week) was predominantly associated with the presence of CYP2C9*2/*3 haplotypes (OR=2.4 [1.3−4.6]) and marginally with age (Table 10, hereinabove). In this study, the VKORC1*2 haplotype was of borderline significance for doses categorized above/below 50 mg/week (OR=0.44 [0.19−0.97]).

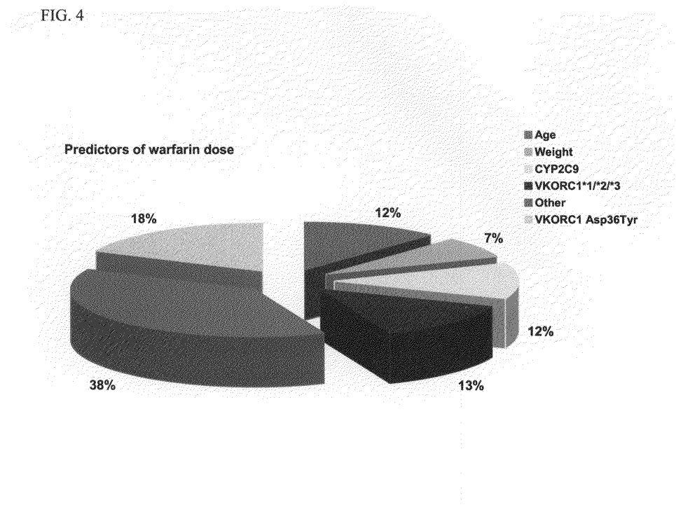

Intermediate dose requirements (20-70 mg/week)—A more detailed analysis of the subgroup of control patients with doses in the range of 20-70 mg/week (n=51), yielded significant associations between dose requirements and age (partial r²=0.10; p=0.007), CYP2C9*1/*2/*3 (partial r²=0.08; p=0.01) and VKORC1*2 (partial r=0.05; p=0.05), as well as the VKORC1 Asp36Tyr polymorphism (partial r²=0.11; p=0.004). Analysis of the entire control group (n=99) demonstrated that the VKORC1 Tyr36Asp polymorphism was the major determinant of warfarin doses (partial r²=0.18) (Table 11, hereinbelow), in addition to the effects of VKORC1*1/*2/*3 and CYP2C9*1*2/*3 (partial r²=0.13 and 0.12, respectively), as well as age and body weight (partial r²=0.12 and 0.07, respectively). This model accounted for a total of 62% of the inter-individual variability in warfarin dose requirements (see also FIG. 4).

TABLE 11
Relative influence of age, weight and various genotypic
markers on dose requirements

| Variable | beta | partial r² | p value |
|---|---|---|---|
| Age | −0.260 | 0.12 | 0.0006 |
| Weight | 0.205 | 0.07 | 0.0073 |
| CYP2C9*1/*2/*3 | −0.257 | 0.12 | 0.0005 |
| VKORC1 Tyr36Asp | 0.372 | 0.18 | <0.0001 |
| VKORC1*1/*2/*3 | −0.277 | 0.13 | 0.0003 |
| EPHX1 (612G→C; | −0.007 | | 0.91 |
| Tyr113His) | | | |
| CALU (R4Q) | 0.079 | | 0.29 |

Table 11: Relative influence of age, weight and various genotypic markers on dose requirements.
The data was analyzed using multiple regression while excluding carriers of VKORC1*4

Asp36Tyr and VKORC1 haplotypes—Haplotype analysis of the unselected warfarin treated group of 99 patients using tag-SNPs of the known VKORC1 haplotypes showed that all carriers of Asp36Tyr had the tag-SNPs of the wild type VKORC1*1, suggesting the possibility of a new configuration. The frequency of this putative Asp36Tyr/*1 haplotype in this group (after verification of Hardy-Weinberg equilibrium) was 4%. The other haplotype frequencies were 41% for VKORC1*2, 37% for VKORC1*3 and 18% VKORC1*4, consistent with the profiles characteristic of Caucasians (Rieder M J, et al., 2005; Geisen C, et al., 2005a), suggesting that the group of 99 patients does reflect an isolated population and thus there is a chance of finding this mutation in other non-Jewish subjects as well. These results suggest that Asp36Tyr polymorphism is a new marker of the high-end of the warfarin dosing range.

Asp36Tyr polymorphism is linked to the VKORC1*1 haplotype—As mentioned in Example 2 hereinabove, the Asp36Tyr polymorphism was found in 15% of Jewish Ethiopian population and in 4% of Ashkenazi Jewish population. Direct sequence analysis of two Ethiopian Jewish individuals exhibiting homozygosity to the 5417T allele (36Y) of VKORC1 (from the control group described in Table 6, Example 2, hereinabove) showed that both were also homozygous for VKORC1*1 haplotype [e.g., exhibited the G allele at position 514 of SEQ ID NO:37 (Tag-SNP of VKORC1*2), the G allele at position 941 of SEQ ID NO:25 (Tag-SNP of VKORC1*3) and the G allele on the + strand (or the C allele on the − strand) at position 256 of SEQ ID NO:38 (Tag-SNP for VKORC1*4)] re-confirming the association between Asp36Tyr and VKORC1*1 haplotype.

Analysis and Discussion

Identification of the genetic factors of inter-individual variability of warfarin dose-response relationship has mainly been directed at variants of the CYP2C9 gene and, more recently, at variants of the VKORC1 gene, encoding a warfarin target protein. A previous study performed by the present inventors (Vecsler M, et al., 2006) suggested that the CALU gene is yet an additional significant modulator of warfarin dose-response. Particularly, the CALU R4Q polymorphism was found to be associated with warfarin resistance. The present study focuses on an exclusive group of warfarin-treated subjects with distinguished dose requirements, in whom an in-depth analysis of the VKORC1, CALU and EPHX1 genes, as well as the two known CYP2C9 genetic variants was performed. The most significant outcome of this study is the finding that the VKORC1 D36Y polymorphism is significantly over-represented in the group of warfarin resistant subjects. Furthermore, concurrent presence of this variation with the other known VKORC1*2 and CYP2C9*3 variations related to lower warfarin doses was still significant, suggesting that the contribution of VKORC1 D36Y to higher warfarin dose is independent and predominant. These results suggest the use of the VKORC1 5417G→T SNP (D36Y) for determining predisposition warfarin resistance.

In one warfarin resistance subject (subject No. 12, Table 4 hereinabove), another rare silent VKORC1 5440C→T (C43C) polymorphism was detected, which was initially described in the original study of VKORC1 mutations associated with the hereditary deficiency of vitamin K-dependent clotting factors type 2 and warfarin resistance [Rost S, 2004 (Supra)] and later reported in a screening study of VKORC1 polymorphisms in the European population [Geisen C, 2005 (Supra)]. More recent functional analysis of site-directed mutants of the seven cystein residues of VKORC1, suggested that the Cystein 43 position is not included in the redox active site that catalyzes vitamin KO reduction (Rost S, et al., 2005, Thromb. Haemost., 94:780-6). However, the involvement of this polymorphism in warfarin resistance is still possible as its' potential consequences should be most probably studied on the level of gene transcription and RNA stability.

A hypothetical model based on studies of animal models of warfarin resistance suggests that CALU is a chaperone that binds to VKOR, GGCX and other integral proteins of the ER membrane. Binding of CALU prevents warfarin from reaching its binding site in VKOR and thus may produce warfarin resistance [Wallin R, 2001 (Supra), Wajih N, 2004 (Supra)]. The previous association study of 100 warfarin-treated subjects supported this notion, as the coding R4Q polymorphisms in CALU gene was found more prevalent in subjects with higher warfarin dose requirements (Vecsler M, et al., 2006). In the present study, homozygotes for this polymorphism (designated as CALU 73A/A genotype) were also more prevalent in the warfarin resistant group. Without being bound with any theory, the effects of VKORC1 5417T and CALU 73A can be additive, as the subject with both these variants had been stabilized by exceptionally high warfarin doses of 140 mg/week. This and the previous study are the only evidence of the effect of CALU on warfarin dose response in subjects. Moreover, both studies consistently suggest that the specific R4Q change at the N-terminal signal peptide domain may have functional implications on the CALU enzyme leading to warfarin resistance. The significance of this polymorphism, in terms of the relative contribution to warfarin dose response and population frequency, should be further explored in a larger series of warfarin-treated subjects and general population subsets.

The CALU 1114A variant was found in both warfarin resistant and warfarin sensitive subjects. The present inventors currently investigate the possible contribution of CALU 1114A genotype to warfarin dose response in an extended series of warfarin-treated subjects.

The present study yielded a novel S78R polymorphism in the coding region of the CALU gene, which was detected in one sensitive subject (subject No. 20, Table 4, hereinabove). The current information of functional and structural features of the CALU protein is limited, however, the idiosyncrasy of S78R (a change from Serine to Arginine) may suggest potential functional implications.

The principal finding of the VKORC1 D36Y polymorphism in warfarin resistant subjects was further validated by the analysis of the unselected group of 99 warfarin-treated subjects. In consistence with the findings of the selected warfarin resistant and sensitive subject (described in Example 1), D36Y was significantly more prevalent in subjects in the upper 75^{th} dose percentile. The effect of D36Y on warfarin dose was also evident by cross-section comparison of the number of D36Y carriers in the resistant group versus the unselected group yielding χ²=13.94, p<0.001.

Further analysis of D36Y in various Jewish ethnic group suggested that D36Y is ethnically stratified and is particularly common in Jewish subjects of Ethiopian origin. Thus, in a subset of 100 individuals of the Ethiopian origin (i.e., 200 chromosomes), the D36Y allele frequency was up to 15%. In addition, three individuals of the Ethiopian Jewish group were found to carry 2 copies of the rare allele (i.e., homozygote to the Y36 variant). D36Y allele was common, although to a lesser extent, in the Ashkenazi Jewish group (4%). Considering that the effect of D36Y is dominant and evident in heterozygous configuration, these findings suggest that a considerable part of the Ethiopian and smaller part of the Ashkenazi populations are predisposed to warfarin resistance, the most prescribed drug for therapy of thromboembolic events.

The influence of ethnicity on warfarin dose requirement has been extensively debated in a number of epidemiological studies, all of which indicated that the African-American subjects are optimized at the highest warfarin doses, namely are warfarin resistant (Blann A, et al., 1999, Br. J. Haematol., 107:207-9; Dang M T, et al., 2005, Ann. Pharmacother., 39:1008-12). These differences could be only partly explained by the respective differences in the frequencies of the known variants of CYP2C9 or drug transporters or other factors involved in warfarin pharmacokinetics [reviewed in El Rouby S, et al., 2004, J. Heart Valve Dis., 13:15-21]. Presently however, VKORC1 has gained a particular attention as the principal genetic modulator of inter-individual and inter-ethnic differences in warfarin response. Two recent studies suggested that warfarin sensitivity, which distinguishes the Chinese patients (Chenhsu R Y, et al., 2000, Ann. Pharmacother., 34:1395-1401), is a reflection of the preponderance of VKORC1 low-dose haplotypes in these population (Yuan H Y, et al., 2005, Hum. Mol. Genet., 14:1745-51; Veenstra D L, et al., 2005, Pharmacogenet Genomics, 15:687-91). The impact of racial differences on the kinetics of dose response or on drug efficacy is not well defined, as few clinical trials take ethnic variation into account. Delineation of the genetic variants underlying the inter-ethnic differences in warfarin response may have profound implications for the efficacy and safety of warfarin use.

Inter-ethnic differences in warfarin maintenance doses have been reported in studies indicating that independent of dietary vitamin K intake, African-American patients require the highest warfarin doses (>5 mg/day), Caucasians have intermediate and Chinese have the lowest warfarin maintenance dose (Absher R K., et al., 2002, Ann Pharmacother 36:1512-7; El Rouby S., et al., 2004, (Supra); Chenhsu R Y, et al., 2000 (Supra); Mestres C A., et al., 2004, J Heart Valve Dis 2004 13:15-21; Yu H. C., et al., 1996, QJM 89:127-35). These differences can be attributed to dietary or genetic variations or common practice of adverse drugs, yet they may have profound implications for the efficacy and safety of warfarin use worldwide. While providing a major indicator of warfarin resistance, the present invention can be implemented in the populations at risk particularly the non-Jewish ethnic groups that are known to require higher warfarin maintenance doses, such as African American and Caucasian. Based on a limited number of studies incorporating no genetic information on VKORC1, it has been suggested that patients of Indian origin are also resistant to warfarin (Zhao F., et al, 2004, Clin Pharmacol Ther 76:210-9; Gan G G., 2003, Int J Hematol. 78:84-6). There is a compelling evidence based on genetic analyses of various mitochondrial DNA (mtDNA) markers of a genetic continuity from Sub-Saharan Africa to the Near East, and furthermore that the migration bottleneck associated with an out of Africa expansion occurred in Ethiopia [reviewed in Lovell A., et al., 2005, Ann Hum Genet 69(Pt 3):275-87]. Complementary to the mtDNA evidence are data from Y-chromosome and autosomal markers, which have also suggested a likely gene flow from Ethiopia into Eurasia, India in particular, and back (reviewed in Cruciani F., et al., 2004, Am J Hum Genet. 74:1014-22; Forster P., et al., 2005, Science 308:965-6; Roychoudhury S., et al., 2001, Hum Genet. 109:339-50). The present findings of a higher prevalence of the “high-dose” VKORC1 D36Y polymorphism in Jews of Ethiopian origin (up to 15% allele frequency) further suggest that this and other polymorphisms associated with warfarin resistance are potentially relevant to the Indian population.

Knowledge of the extent to which these factors affect anticoagulation response could help in the prediction of a more individualized loading and maintenance warfarin dose for a safer anticoagulation therapy. In the present study and in a previous study performed by the present inventors (Vecsler M, et al., 2006), several genetic variations were characterized in the major modulators of warfarin pharmacokinetics and pharmacodynamics that significantly contribute to warfarin dose response. These naturally occurring genetic variants may be included in a comprehensive framework of a prospective study testing genetic predisposition to warfarin dose response adjusted for confounding factors. Thus, the teachings of the present invention form the basis for a more inclusive and accurate dosing algorithm enabling the prediction of initial warfarin requirement and the development of a novel individualized dosing regimen that may potentially decrease the rates of hemorrhagic and thrombotic complications with the use of coumarin derivatives such as warfarin.

Concerns regarding oral anticoagulation focus on the two extremes of individual sensitivity to warfarin: the very sensitive patients with an increased risk of bleeding and at the other end, warfarin resistant patients susceptible to under-anticoagulation with increased risk of thrombosis. The main finding of this study adds another layer to the understanding of contribution of the genetic factors on warfarin dose requirements. While, CYP2C9 and VKORC1 haplotypes enable classification of warfarin dose requirements as low (7-20 mg/week per week) or intermediate (20-70 mg/week), the VKORC1 Asp36Tyr polymorphism provides a new marker that is strongly and specifically indicative of high (>70 mg/week) warfarin dose requirements. In addition, this polymorphism contributed 18% to the explanatory capacity of the variability of warfarin dose-response relationship, above the effects of CYP2C9 and known VKORC1 haplotypes. This revealed total variance explained to 62%. The dose-increasing effect of Asp36Tyr was dominant over the dose-reducing effect of CYP2C9*2, *3, and VKORC1*2 haplotypes. These findings suggest that future models for prediction of warfarin dose requirements should include the new Asp36Tyr marker as well as the presently known CYP2C9 and VKORC1 markers. With the inclusion of Asp36Tyr in the prediction model, the EPHX1 Tyr113H is polymorphism was not significant.

The efficacy and safety of warfarin may be profoundly affected by ethnic differences. Dose requirements have been shown to vary across ethnic groups, with African-Americans patients requiring higher maintenance doses and Japanese and Chinese lower doses compared to Caucasians (Absher et al. 2002; Dang M T, et al., 2005; Gan G G, et al., 2003; Schwarz U I, et al., 2006). The distribution of CYP2C9 genetic variants across ethnic groups explains these differences only partially (Scordo M G et al., 2001; Takahashi H, et al., 2003; Zhao F, et al., 2004). Inclusion of the newer VKORC1 variants has furthered understanding these inter-ethnic differences (Yuan H Y, et al., 2005; Veenstra D L, et al., 2005; Mushiroda T., et al., 2006; Takahashi H, et al., 2006). The new Asp36Tyr marker of warfarin resistance, which probably evolved on the background of the wild type VKORC1*1 haplotype, may serve to explain epidemiological observations of increased dose requirements in other ethnic non-Jewish ethnic groups, e.g. African Americans, for whom VKORC1*1 is characteristic (Geisen C, et al., 2005a).

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### REFERENCES

### Additional References are Cited in Text

1. Absher et al., Ann Pharmacother 2002, 36:1512-7;
2. Bodin L., et al., 2005, Blood, 106: 135-40;
3. D'Andrea G, et al., 2005, Blood, 105: 645-9;
4. Dang et al., 2005, Ann Pharmacother 39:1008-11;
5. Gan G G., 2003, Int J Hematol. 78:84-6;
6. Geisen C, et al., 2005a, Thromb Haemost, 94:773-9;
7. Geisen C, et al., 2005b, Journal of Thrombosis and Haemostasis; Volume 3, Supplement 1: Abstract No. P0056. A Novel Mutation (Asp36Tyr) in the Vitamin K Epoxide Reductase Complex Subunit 1 (VKORC1) Causes Moderately Increased Coumarin Doses;
8. Harrington D J, et al., 2005, Thromb. Haemost., 93:23-6;
9. Higashi M K et al 2002, JAMA 287:1690-8;
10. Loebstein R, et al., 2005, Clin. Pharmacol. Ther., 77:365-72;
11. Loebstein R., et al., 2007, Blood, 109 (6): 2477-2480;
12. Mushiroda T., et al., 2006, J. Hum. Genet. 2006; 51(3):249-53. Association of VKORC1 and CYP2C9 polymorphisms with warfarin dose requirements in Japanese patients;
13. Reitsma P H, et al., 2005, PLoS Med 2:e312;
14. Rieder M J., et al., 2005, N. Engl. J. Med., 352: 2285-93;
15. Rost S., et al., 2004 (Nature, 427:537-41);
16. Schelleman H., et al., 2007. Clin Pharmacol Ther. February 28; [Epub ahead of print];
17. Schwarz U I, Stein C M. Genetic determinants of dose and clinical outcomes in patients receiving oral anticoagulants. Clin Pharmacol Ther. 2006; 80:7-12;
18. Sconce et al., 2005, Blood, 106: 2329-33;
19. Scordo M G, Aklillu E, Yasar U, et al. Genetic polymorphism of cytochrome P450 2C9 in a Caucasian and a black African population. Br J Clin Pharmacol. 2001; 52:447-50.
20. Shikata E, et al., 2004, Blood, 103:2630-5;
21. Takahashi et al., 2003, Clin. Pharmacology and Therapeutics 73:253-263;
22. Takahashi H., et al., 2006, Pharmacogenetics and Genomics 16:101-110;
23. Vecsler M, et al., 2006, Thromb Haemost. 95(2):205-11. Combined genetic profiles of components and regulators of the vitamin K-dependent γ-carboxylation system affect individual sensitivity to warfarin;
24. Veenstra D L., et al., 2005, Pharmacogenet Genomics 15: 687-91;
25. Wadelius M, et al., 2005, Pharmacogenomics J., 5:262-70;
26. Wadelius M et al 2007, Hum Genet 121:23-34;
27. Wajih N, et al., 2004, J. Biol. Chem., 279:25276-83;
28. Wallin R, et al., 2001, FASEB J., 15:2542-4;
29. Yuan H-Y, et al., 2005, Hum. Mol. Genet. 14: 1745-1751;
30. Zhao et al., 2004, Clin Pharmacology and Therapeutics 76:210-219;

1. A method of determining if an individual is predisposed to coumarin resistance, the method comprising determining in a sample of the individual a presence or an absence, in a homozygous or a heterozygous form of a thymidine nucleotide-containing allele at position 5417 of a VKORC1 polynucleotide as set forth in SEQ ID NO:25 and/or a tyrosine residue-containing polymorph at position 36 of a VKORC1 polypeptide as set forth in SEQ ID NO:28, wherein a presence of said thymidine nucleotide-containing allele at position 5417 of said VKORC1 polynucleotide and/or said tyrosine residue-containing polymorph at position 36 of said VKORC1 polypeptide is indicative of predisposition to coumarin resistance, thereby determining if the individual is predisposed to coumarin resistance.

2. A kit for determining if an individual is predisposed to coumarin resistance, the kit comprising at least one reagent for determining a presence or an absence in a homozygous or a heterozygous form of a thymidine nucleotide-containing allele at position 5417 of a VKORC1 polynucleotide as set forth in SEQ ID NO:25 and/or a tyrosine residue-containing polymorph at position 36 of a VKORC1 polypeptide as set forth in SEQ ID NO:28 and instructions for use in determining if an individual is predisposed to coumarin resistance, wherein a presence of said thymidine nucleotide-containing allele at position 5417 of said VKORC1 polynucleotide and/or said tyrosine residue-containing polymorph at position 36 of said VKORC1 polypeptide is indicative of predisposition to coumarin resistance.

3. A method of predicting a responsiveness of an individual to coumarin treatment, comprising detecting in a sample of the individual a presence or an absence, in a homozygous or a heterozygous form of a thymidine nucleotide-containing allele at position 5417 of a VKORC1 polynucleotide as set forth in SEQ ID NO:25 and/or a tyrosine residue-containing polymorph at position 36 of a VKORC1 polypeptide as set forth in SEQ ID NO:28, thereby predicting the responsiveness of the individual to coumarin treatment.

4. (canceled)

5. A method of determining if an individual is suitable for genotype analysis of VKORC1 D36Y-related coumarin resistance, comprising determining in a sample of the individual a presence or an absence, in a homozygous or a heterozygous form of a VKORC1*1 haplotype, wherein said presence of said VKORC1*1 haplotype is indicative of the individual being suitable for genotype analysis of the VKORC1 D36Y-related coumarin resistance.

6. A kit for determining if an individual is suitable for genotype analysis of VKORC1 D36Y-related coumarin resistance, the kit comprising at least one reagent for determining a presence or an absence in a homozygous or a heterozygous form of a VKORC1*1 haplotype and instructions for use in determining if an individual is suitable for genotype analysis of VKORC1 D36Y-related coumarin resistance, wherein said presence of said VKORC1*1 haplotype is indicative of the individual being suitable for genotype analysis of VKORC1 D36Y-related coumarin resistance.

7. The method of claim 1, wherein said coumarin is warfarin.

8. The method of claim 1, wherein the individual is predisposed to thromboembolism.

9-12. (canceled)

13. The kit of claim 2, wherein said at least one reagent is at least one oligonucleotide capable of specifically hybridizing with a thymidine nucleotide-containing allele or a guanine nucleotide-containing allele at position 5417 of said VKORC1 polynucleotide.

14. The method of claim 1, wherein said determining said presence or absence of said thymidine nucleotide-containing allele at position 5417 of said VKORC1 polynucleotide or said VKORC1*1 haplotype is effected by a method selected from the group consisting of: DNA sequencing, restriction fragment length polymorphism (RFLP analysis), allele specific oligonucleotide (ASO) analysis, Denaturing/Temperature Gradient Gel Electrophoresis (DGGE/TGGE), Single-Strand Conformation Polymorphism (SSCP) analysis, Dideoxy fingerprinting (ddF), pyrosequencing analysis, acycloprime analysis, Reverse dot blot, GeneChip microarrays, Dynamic allele-specific hybridization (DASH), Peptide nucleic acid (PNA) and locked nucleic acids (LNA) probes, TaqMan, Molecular Beacons, Intercalating dye, FRET primers, AlphaScreen, SNPstream, genetic bit analysis (GBA), Multiplex minisequencing, SNaPshot, MassEXTEND, MassArray, GOOD assay, Microarray miniseq, arrayed primer extension (APEX), Microarray primer extension, Tag arrays, Coded microspheres, Template-directed incorporation (TDI), fluorescence polarization, Colorimetric oligonucleotide ligation assay (OLA), Sequence-coded OLA, Microarray ligation, Ligase chain reaction, Padlock probes, Rolling circle amplification, Sequenom Mass Spectrograph and Invader assay.

15. The kit of claim 2, wherein said at least one reagent is an antibody capable of differentially binding at least one polymorph of an Aspartic acid residue-containing polymorph or a said tyrosine residue-containing polymorph at position 36 of said VKORC1 polypeptide.

16. The method of claim 1, wherein said determining said presence or absence of said tyrosine residue-containing polymorph is effected by an antibody capable of differentially binding at least one polymorph of an Aspartic acid residue-containing polymorph or a said tyrosine residue-containing polymorph at position 36 of said VKORC1 polypeptide.

17. The method of claim 14, wherein said sample of the individual is a DNA sample.

18. The method of claim 16, wherein said sample of the individual is a protein sample.

19. The method of claim 1, wherein the individual carries the VKORC1*1 haplotype.

20. The method of claim 1, wherein the individual is of a population selected from the group consisting of an African population, an African American population, a Jewish Ethiopian population, an Ashkenazi Jewish population, Caucasian population and an Indian population.

21. The method of claim 5, wherein said VKORC1*1 haplotype comprises the guanine nucleotide-containing allele at position 514 of SEQ ID NO:37, the guanine nucleotide-containing allele at position 9041 of SEQ ID NO:25 and the guanine nucleotide-containing allele at position 256 of SEQ ID NO:38.

22. The kit of claim 6, wherein said at least one reagent is at least one oligonucleotide capable of specifically hybridizing with a guanine nucleotide-containing allele at position 514 of SEQ ID NO:37, a guanine nucleotide-containing allele at position 9041 of SEQ ID NO:25 and/or a guanine nucleotide-containing allele at position 256 of SEQ ID NO:38.

23. The method of claim 3, wherein the individual carries the VKORC1*1 haplotype.

24. The method of claim 6, wherein said VKORC1*1 haplotype comprises the guanine nucleotide-containing allele at position 514 of SEQ ID NO:37, the guanine nucleotide-containing allele at position 9041 of SEQ ID NO:25 and the guanine nucleotide-containing allele at position 256 of SEQ ID NO:38.

25. The method of claim 19, wherein said VKORC1*1 haplotype comprises the guanine nucleotide-containing allele at position 514 of SEQ ID NO:37, the guanine nucleotide-containing allele at position 9041 of SEQ ID NO:25 and the guanine nucleotide-containing allele at position 256 of SEQ ID NO:38.

26. The method of claim 3, wherein the individual is of a population selected from the group consisting of an African population, an African American population, a Jewish Ethiopian population, an Ashkenazi Jewish population, Caucasian population and an Indian population.

27. The method of claim 3, wherein said coumarin is warfarin.
